SEQUENCE LISTING (12 sequences)

SEQ ID NO: 1 - DNA, 52 bp, Homo sapiens
  misc_feature 27: n=c or t
cttgaaggagaaggtgtctgcgggagncgatttcatcatcacgcagcttttc

SEQ ID NO: 2 - DNA, 52 bp, Homo sapiens
  misc_feature 27: n=a or g
ggaagaatatgaagatattagacaggnccattatgagtctctcaaggtaagt

SEQ ID NO: 3 - DNA, 52 bp, Homo sapiens
  misc_feature 27: n=c or t
aagctgagaacatcaagaagttcttanagtaagtacatcctcgaaagtttat

SEQ ID NO: 4 - DNA, 30 bp, Artificial Sequence
  laboratory-synthesized DNA primers
acgttggatggaagcacttgaaggagaagg

SEQ ID NO: 5 - DNA, 30 bp, Artificial Sequence
  laboratory-synthesized DNA primers
acgttggatgagcctcaaagaaaagctgcg

SEQ ID NO: 6 - DNA, 16 bp, Artificial Sequence
  laboratory-synthesized DNA primers
aaggtgtctgcgggag

SEQ ID NO: 7 - DNA, 30 bp, Artificial Sequence
  laboratory-synthesized DNA primers
acgttggatgctttgaggaaatcatggaag

SEQ ID NO: 8 - DNA, 30 bp, Artificial Sequence
  laboratory-synthesized DNA primers
acgttggatgtctaccacttaccttgagag

SEQ ID NO: 9 - DNA, 23 bp, Artificial Sequence
  laboratory-synthesized DNA primers
agaatatgaagatattagacagg

SEQ ID NO: 10 - DNA, 30 bp, Artificial Sequence; identical to SEQ ID NO: 7
  laboratory-synthesized DNA primers

SEQ ID NO: 11 - DNA, 30 bp, Artificial Sequence
  laboratory-synthesized DNA primers
acgttggatggtccatataaactttcgagg

SEQ ID NO: 12 - DNA, 28 bp, Artificial Sequence
  laboratory-synthesized DNA primers
taaagctgagaacatcaagaagttctta

(19) United States
(12) Patent Application Publication
(10) Pub. No.: US 2014/0088035 A1
(43) Pub. Date: Mar. 27, 2014

(54) TREATING SCHIZOPHRENIA

(71) Applicants: Donald C. Goff, New York, NY (US); Joshua Roffman, Newton, MA (US)

(72) Inventors: Donald C. Goff, New York, NY (US); Joshua Roffman, Newton, MA (US)

(73) Assignee: THE GENERAL HOSPITAL CORPORATION, Boston, MA (US)

(21) Appl. No.: 13/885,337

(22) PCT Filed: Dec. 2, 2011
(86) PCT No.: PCT/US2011/063130

§ 371 Date: Dec. 2, 2013

Related U.S. Application Data
(60) Provisional application No. 61/419,742, filed on Dec. 3, 2010.

(51) Int. Cl. C12Q 1/68; A61K 31/7056; A61K 31/519

(52) U.S. Cl. CPC C12Q 1/6883; A61K 31/519; A61K 31/7056
USPC 514/52; 514/249; 435/6.11

(57) ABSTRACT

The specification provides methods of treating a subject suffering from a negative symptom of schizophrenia and methods of determining whether a subject is suffering from or at risk for developing a negative symptom of schizophrenia.

### CROSS-REFERENCE TO RELATED APPLICATION

This application claims priority under 35 U.S.C. §119(e) to U.S. Provisional Patent Application Ser. No. 61/419,742, filed on Dec. 3, 2010, the entire contents of which are hereby incorporated by reference in its entirety.

### FEDERALLY SPONSORED RESEARCH OR DEVELOPMENT

This invention was made with Government support under Grant Number R01 MH070831-A2 awarded by National Institute of Mental Health. The Government has certain rights in the invention.

### TECHNICAL FIELD

The claimed methods relate to genetic markers of schizophrenia and methods of use thereof.

### BACKGROUND

Schizophrenia is a mental disorder characterized by a disintegration of the process of thinking and of emotional responsiveness. It most commonly manifests as auditory hallucinations, paranoid or bizarre delusions, or disorganized speech and thinking, and it is accompanied by significant social or occupational dysfunction. The onset of symptoms typically occurs in young adulthood, with a global lifetime prevalence of around 1.5 percent. Diagnosis is based on the patient's self-reported experiences and observed behavior.

Genetics, early environment, neurobiology, psychological and social processes appear to be important contributory factors; some recreational and prescription drugs appear to cause or worsen symptoms. Current psychiatric research is focused on the role of neurobiology, but this inquiry has not isolated a single organic cause. As a result of the many possible combinations of symptoms, there is debate about whether the diagnosis represents a single disorder or a number of discrete syndromes. Unusually high dopamine activity in the mesolimbic pathway of the brain has been found in people with schizophrenia. The mainstay of treatment is antipsychotic medication; this type of drug primarily works by suppressing dopamine activity. Psychotherapy and vocational and social rehabilitation are also important. In more serious cases, where there is risk to self and others, involuntary hospitalization may be necessary.

The disorder is thought mainly to affect cognition, but it also usually contributes to chronic problems with behavior and emotion. People with schizophrenia are likely to have additional (comorbid) conditions, including major depression and anxiety disorders; the lifetime occurrence of substance abuse is around 40 percent. Social problems, such as long-term unemployment, poverty and homelessness, are common. Further, the average life expectancy of people with the disorder is 10 to 12 years less than those without, due to increased physical health problems and a higher suicide rate (Palmer et al., Archives of General Psychiatry 2005; 62:247-53). Therefore, more effective methods of determining whether a subject is suffering from or at risk for developing a negative symptom of schizophrenia and methods of selecting an appropriate treatment for a subject suffering from a negative symptom of schizophrenia are desirable.

### SUMMARY

Methods to predict treatment response to vitamin supplementation and other treatments for symptoms of schizophrenia are described. The present specification provides a panel of single nucleotide polymorphism (SNP) biomarkers for predicting the response to treatment. In one aspect, the methods described herein feature methods of selecting an appropriate treatment for a subject based on a presence of one or more alleles at rs1801133, rs1805087, and rs202676 in genomic DNA.

In one aspect, the methods described herein feature methods of treating a subject, e.g., a human, diagnosed as having a negative symptom of schizophrenia, e.g., apathy, impoverished speech, flattened affect, social withdrawal, or any combination thereof, are provided. The methods include determining the presence of one or more alleles at rs1801133, rs1805087, and rs202676 in a sample comprising genomic DNA from the subject, e.g., plasma or whole blood, selecting a treatment for the subject based on the presence of the one or more alleles, and treating the subject with the selected treatment.

In some embodiments, the selected treatment includes prescribing or administering folate and/or vitamin B12 to the subject.

In one embodiment, if a “T” at rs1801133, an “A” at rs1805087, or a “T” at rs202676 is present, then a treatment comprising administering folate and/or vitamin B12 to the subject is selected. In some embodiments, if a “T” at rs1801133, an “A” at rs1805087, and a “T” at rs202676 are present, then a treatment comprising prescribing or administering folate and/or vitamin B12 to the subject is selected.

In some embodiments, the methods include determining the presence of six alleles, wherein the six alleles consist of two alleles at each of rs1801133, rs1805087, and rs202676. If a “T” at rs1801133, an “A” at rs1805087, and a “T” at rs202676 are present, and one or more additional alleles are a “T” at rs1801133, an “A” at rs1805087, or a “T” at rs202676, then a treatment comprising prescribing or administering folate and/or vitamin B12 to the subject is selected.

In some examples, if two alleles are a “T” at rs1801133 and two alleles are an “A” at rs1805087, then a treatment comprising prescribing or administering folate and/or vitamin B12 to the subject is selected. In one embodiment, if two alleles are a “T” at rs1801133 and two alleles are an “A” at rs1805087, and one or more additional alleles is a “T” at rs202676, then a treatment comprising prescribing or administering folate and/or vitamin B12 to the subject is selected.

In one embodiment, if a “T” at rs1801133, an “A” at rs1805087, and a “T” at rs202676 are present, and two or more additional alleles are a “T” at rs1801133, an “A” at rs1805087, or a “T” at rs202676, then a treatment comprising prescribing or administering folate and/or vitamin B12 to the subject is selected.

In some examples, if two alleles are a “T” at rs1801133, two alleles are an “A” at rs1805087, and two alleles are a “T” at rs202676, then a treatment comprising prescribing or administering folate and/or vitamin B12 to the subject is selected.

In one embodiment, if a “T” at rs1801133, an “A” at rs1805087, or a “T” at rs202676 is not present, then a treatment comprising a psychosocial intervention is selected. In one embodiment, if one or more, e.g., two or more, three or more, four or more, five or more, or six, of a “T” at rs1801133, an “A” at rs1805087, and a “T” at rs202676 is not present, then a treatment comprising a psychosocial intervention is selected.

In one aspect, the methods described herein include assaying for the presence of one or more alleles at rs1801133, rs1805087, and rs202676 in a biological sample comprising genomic DNA from a subject diagnosed as having a negative symptom of schizophrenia, e.g., apathy, impoverished speech, flattened affect, social withdrawal, or any combination thereof, and transmitting to a recipient, e.g., health care provider, medical caregiver, physician, and nurse, a report on the presence of the one or more alleles.

In some embodiments, the biological sample comprising genomic DNA can be, e.g., plasma or whole blood, from the subject, e.g., a human.

In one embodiment, the methods include selecting a treatment for reducing the negative symptom in the subject based on the presence of the one or more alleles. In some embodiments, the selected treatment includes prescribing or administering folate and/or vitamin B12 to the subject.

In one embodiment, if a “T” at rs1801133, an “A” at rs1805087, or a “T” at rs202676 is present, then a treatment comprising administering folate and/or vitamin B12 to the subject is selected. In some embodiments, if a “T” at rs1801133, an “A” at rs1805087, and a “T” at rs202676 are present, then a treatment comprising prescribing or administering folate and/or vitamin B12 to the subject is selected.

In some embodiments, the methods include determining the presence of six alleles, wherein the six alleles consist of two alleles at each of rs1801133, rs1805087, and rs202676. If a “T” at rs1801133, an “A” at rs1805087, and a “T” at rs202676 are present, and one or more additional alleles are a “T” at rs1801133, an “A” at rs1805087, or a “T” at rs202676, then a treatment comprising prescribing or administering folate and/or vitamin B12 to the subject is selected.

In some examples, if two alleles are a “T” at rs1801133 and two alleles are an “A” at rs1805087, then a treatment comprising prescribing or administering folate and/or vitamin B12 to the subject is selected. In one embodiment, if two alleles are a “T” at rs1801133 and two alleles are an “A” at rs1805087, and one or more additional alleles is a “T” at rs202676, then a treatment comprising prescribing or administering folate and/or vitamin B12 to the subject is selected.

In one embodiment, if a “T” at rs1801133, an “A” at rs1805087, and a “T” at rs202676 are present, and two or more additional alleles are a “T” at rs1801133, an “A” at rs1805087, or a “T” at rs202676, then a treatment comprising prescribing or administering folate and/or vitamin B12 to the subject is selected.

In some examples, if two alleles are a “T” at rs1801133, two alleles are an “A” at rs1805087, and two alleles are a “T” at rs202676, then a treatment comprising prescribing or administering folate and/or vitamin B12 to the subject is selected.

In yet another aspect, a plurality of polynucleotides bound to a solid support are provided. Each polynucleotide of the plurality selectively hybridizes to one or more SNP alleles selected from the group consisting of rs1801133, rs1805087, and rs202676.

In some embodiments, the plurality of polynucleotides comprise SEQ ID NOs:4, 5, 6, 7, 8, 9, 10, 11, 12, and any combination thereof.

In some aspects, the specification provides nucleotide sequences, e.g., polynucleotides comprising the sequences of SEQ ID NOs:4, 5, 6, 7, 8, 9, 10, 11, and 12, to detect a presence of one or more alleles at rs1801133, rs1805087, and rs202676.

As used herein, the term “schizophrenia” refers to a psychiatric disorder that includes at least one of the following: delusions, hallucinations, disorganized speech, grossly disorganized or catatonic behavior, or negative symptoms (e.g., apathy, impoverished speech, flattened affect, and social withdrawal). Patients can be diagnosed as schizophrenic using the DSM-IV criteria (APA, 1994, Diagnostic and Statistical Manual of Mental Disorders (Fourth Edition), Washington, D.C.). Subjects can be diagnosed as having a negative symptom of schizophrenia by a health care provider, medical caregiver, physician, nurse, family member, or acquaintance, who recognizes, appreciates, acknowledges, determines, concludes, opines, or decides that the subject has a negative symptom of schizophrenia.

If desired, one can measure negative and/or positive and/or cognitive symptom(s) of schizophrenia before and after treatment of the subject. A reduction in such a symptom indicates that the subject's condition has improved. Improvement in the symptoms of schizophrenia can be assessed using the Scales for the Assessment of Negative Symptoms (SANS), Iowa City, Iowa and Kay et al., 1987, Schizophrenia Bulletin 13:261-276) or Positive and Negative Syndrome Scale (PANSS) (see, e.g., Andreasen, 1983).

As used herein, the term “psychosocial intervention” refers to interactions with clinical staff or with other patients in a group setting, which could consist of psychotherapy, group therapy, social skills training, vocational rehabilitation or other interactive treatments or rehabilitation activities.

As used herein, an “allele” is one of a pair or series of genetic variants of a polymorphism at a specific genomic location. A “schizophrenia susceptibility allele” is an allele that is associated with increased susceptibility of developing schizophrenia.

As used herein, a “haplotype” is one or a set of signature genetic changes (polymorphisms) that are normally grouped closely together on the DNA strand, and are usually inherited as a group; the polymorphisms are also referred to herein as “markers.” A haplotype is information regarding the presence or absence of one or more genetic markers in a given chromosomal region in a subject. A haplotype can consist of a variety of genetic markers, including indels (insertions or deletions of the DNA at particular locations on the chromosome); SNPs in which a particular nucleotide is changed; microsatellites; and minisatellites.

As used herein, an “based on” refers to taking the presence of one or more alleles, e.g., at rs1801133, rs1805087, and rs202676, into consideration or accounting for the presence of one or more alleles, e.g., at rs1801133, rs1805087, and rs202676.

“Linkage disequilibrium” refers to when the observed frequencies of haplotypes in a population does not agree with haplotype frequencies predicted by multiplying together the frequency of individual genetic markers in each haplotype.

The term “chromosome” as used herein refers to a gene carrier of a cell that is derived from chromatin and comprises DNA and protein components (e.g., histones). The conventional internationally recognized individual human genome chromosome numbering identification system is employed herein. The size of an individual chromosome can vary from one type to another with a given multi-chromosomal genome and from one genome to another. In the case of the human genome, the entire DNA mass of a given chromosome is usually greater than about 100,000,000 base pairs. For example, the size of the entire human genome is about 3×10⁹ base pairs.

The term “gene” refers to a DNA sequence in a chromosome that codes for a product (either RNA or its translation product, a polypeptide). A gene contains a coding region and includes regions preceding and following the coding region (termed respectively “leader” and “trailer”). The coding region is comprised of a plurality of coding segments (“exons”) and intervening sequences (“introns”) between individual coding segments.

The term “probe” refers to an oligonucleotide. A probe can be single stranded at the time of hybridization to a target. As used herein, probes include primers, i.e., oligonucleotides that can be used to prime a reaction, e.g., a PCR reaction.

Unless otherwise defined, all technical terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWING

FIG. 1 is a schematic diagram of the folate metabolic pathway. THF: tetrahydrofolate; Met: methionine; SAM: S-adenosylmethionine; SAH: S-adenosylhomocysteine; and Hcy: homocysteine.

FIG. 2 is a series of three bar graphs showing that three genetic variants in the folate metabolic pathway significantly predict negative symptom severity. Error bars indicate standard error.

FIG. 3 is a scatter plot showing effects of cumulative MTHFR 677T, MTR 2756A, and FOLH1 484C risk allele load on negative symptom severity. Each marker represents a single subject. Markers are slightly jittered to avoid overlap.

FIG. 4A is a series of two bar graphs depicting distribution of subjects by risk allele load. FIG. 4B is series of three scatter plots showing the relationship between negative symptom severity and serum folate, stratified by risk allele load.

FIG. 5A is a series of seven line graphs showing overall effect of folate supplementation versus placebo on change in negative symptoms (SANS) scores as well as treatment effects stratified by MTHFR 677T, MTR 2756A, and FOLH1 484T SNPs. A score below zero indicates an improvement from baseline. Broken lines indicate hypofunctional variants. FIG. 5B is a scatter plot showing an improvement in negative symptoms over 16 weeks of folate supplementation as correlated with folate treatment score, which is calculated based on the total number of folate alleles (0, 1, or 2) that an individual possesses across all three SNPs.

FIG. 6 is a scatter plot showing that RBC folate levels differed at baseline as a function of FOLH1 genotype, and C/C patients who received folate and vitamin B12 did not catch up until week 8.

FIG. 7 is a series of two bar graphs showing genotype frequency variation by ethnicity. To examine additive effects of alleles across FOLH1, MTHFR, and MTR in the current cohort, only Caucasian subjects were studied, diminishing the likelihood of population stratification artifact.

### DETAILED DESCRIPTION

Approximately 30 percent of patients with schizophrenia suffer from treatment-resistant psychotic symptoms which can produce substantial distress, result in hospitalization and disrupt attempts to function in school or work. In particular, negative symptoms remain largely treatment refractory and also are a major contributor to disability in people with schizophrenia. While the atypical antipsychotics have demonstrated some benefit over the conventional agents for negative symptoms, it is unclear the degree to which primary negative symptoms respond, particularly in patients with the deficit syndrome. Vitamin supplementation with folate and vitamin B12 represents a safe and inexpensive approach that can improve outcomes for patients with negative symptoms.

The methods described herein are based, at least in part, on markers that are associated with negative symptoms of schizophrenia and with response to folate treatment (Table 1). Analysis provided evidence of an association of the disclosed SNPs and symptoms of this disease. A SNP occurs at a polymorphic site occupied by a single nucleotide, which is the site of variation between allelic sequences. The site is usually preceded by and followed by highly conserved sequences of the allele (e.g., sequences that vary in less than 1/100 or 1/1000 members of the populations). A SNP usually arises due to substitution of one nucleotide for another at the polymorphic site. A transition is the replacement of one purine by another purine or one pyrimidine by another pyrimidine. A transversion is the replacement of a purine by a pyrimidine or vice versa. Single nucleotide polymorphisms can also arise from a deletion of a nucleotide or an insertion of a nucleotide relative to a reference allele. Typically the polymorphic site is occupied by a base other than the reference base. For example, where the reference allele contains the base “C” at the polymorphic site, the altered allele can contain a “T,” “G,” or “A” at the polymorphic site.

TABLE 1
SNPs Associated with Negative Symptoms of Schizophrenia and
Treatment Response to Folate and/or Vitamin B12

| | | | Risk | Folate |
|---|---|---|---|---|
| SNP | Location | Sequence | Allele | Allele |
| MTHFR | 1p36 | CTTGAAGGAGAAGGTGTCTGCGGGAG[C/T] | T | T |
| rs1801133 | | CGATTTCATCATCACGCAGCTTTTC | | |
| | | (SEQ ID NO: 1) | | |
| MTR | 1q43 | GGAAGAATATGAAGATATTAGACAGG[A/G] | A | A |
| rs1805087 | | CCATTATGAGTCTCTCAAGGTAAGT | | |
| | | (SEQ ID NO: 2) | | |
| FOLH1 | 11p11 | AAGCTGAGAACATCAAGAAGTTCTTA[C/T] | C | T |
| rs202676 | | AGTAAGTACATCCTCGAAAGTTTAT | | |
| | | (SEQ ID NO: 3) | | |

A series of SNP risk alleles have been identified that are associated with negative symptoms of schizophrenia. The presence of one or more of SNP risk alleles, e.g., two, three, four, five, or six risk alleles described in Table 1, can be used to determine whether a subject is suffering from or at risk for developing a negative symptom of schizophrenia. The presence of one or more of SNP folate alleles, e.g., two, three, four, five, or six folate alleles described in Table 1, can be used select a treatment, e.g., folate and/or vitamin B12, for a subject suffering from a negative symptom of schizophrenia. The SNP genotypes (identified by their SNP site and alleles) are depicted in Table 1. Further information on the SNPs can be obtained from, for example, the National Center for Biotechnology Information Entrez Single Nucleotide Polymorphism database that is accessible via the Internet. Genetic variation throughout the folate metabolic pathway contributes to negative symptoms in schizophrenia. Missense variants in three genes, MTHFR, MTR, and FOLH1, are independently associated with negative symptom scores. Moreover, the specification provides evidence of a cumulative effect of risk and folate variants in MTHFR, MTR, and FOLH1, where patients who carry more than three, e.g., four, five, or six, risk alleles across the three genes exhibited a stronger inverse relationship between serum folate level and negative symptom scores.

### Methods for Determining Susceptibility to a Negative Symptom of Schizophrenia

Described herein are a variety of methods of determining whether a subject is suffering from or at risk for developing a negative symptom of schizophrenia. An increased susceptibility to a negative symptom of schizophrenia exists if a subject has an allele or a haplotype associated with an increased susceptibility to a negative symptom of schizophrenia, i.e., a “risk allele,” as described in Table 1. Ascertaining or assaying whether the subject has such a risk allele or a haplotype is included in the concept of determining susceptibility to a negative symptom of schizophrenia. Such determination is useful, for example, for purposes of diagnosis, treatment selection (e.g., of new or different treatments), and genetic counseling. Thus, the methods described herein can include assaying or detecting an allele or a haplotype associated with an increased susceptibility to a negative symptom of schizophrenia as described herein for the subject.

### Methods of Treating a Subject Having a Negative Symptom of Schizophrenia

Described herein are a variety of methods of treating a subject having a negative symptom of schizophrenia. A decrease in negative symptoms of schizophrenia in response to folate and/or vitamin B12 treatment results if a subject has an allele or a haplotype associated with a “folate allele,” as described in Table 1. Ascertaining or assaying whether the subject has such a folate allele or a haplotype is included in the concept of treating a subject having a negative symptom of schizophrenia. Such determination is useful, for example, for purposes of diagnosis, treatment selection (e.g., folate and/or vitamin B12, and new or different treatments), and genetic counseling. Thus, the methods described herein can include assaying or detecting an allele or a haplotype associated with a decrease in negative symptoms of schizophrenia in response to folate and/or vitamin B12 treatment as described herein for the subject.

Also described herein are a variety of methods for determining a subject who, having a negative symptom of schizophrenia, will respond positively to a placebo treatment. A decrease in negative symptoms of schizophrenia in response to placebo treatment results if the subject has less than six alleles or haplotypes, e.g., five, four, three, two, or one, “folate allele,” as described in Table 1. Ascertaining or assaying whether the subject has such a folate allele or a haplotype is included in the concept of determining subjects who respond to placebo. Such determination is useful, for example, for purposes of clinical trials, interpreting results, diagnosis, treatment selection (e.g., of new or different treatments), and genetic counseling. Thus, the methods described herein can include assaying or detecting an allele or a haplotype associated with a response to a placebo. The clinical implication is that subjects having a negative symptom of schizophrenia, who have a folate treatment score (i.e., (0, 1, or 2 copies of MTHFR 677T)+(0, 1, or 2 copies of FOLH1 484T)+(0, 1, or 2 copies of MTR 2756A)=0 to 6 total folate alleles) of less than six alleles or haplotypes, e.g., five, four, three, two, or one, “folate allele,” as described in Table 1, are best treated with a psychosocial intervention, e.g., interactions with clinical staff or with other patients in a group setting, which could consist of psychotherapy, group therapy, social skills training, vocational rehabilitation or other interactive treatments or rehabilitation activities.

Methods of Determining the Presence or Absence of an Allele or a Haplotype Associated with Schizophrenia

The methods described herein include determining the presence or absence of alleles or haplotypes associated with schizophrenia. In some embodiments, an association with schizophrenia is determined by the presence of a shared haplotype between the subject and an affected reference individual, e.g., a first or second-degree relation of the subject, and the absence of the haplotype in an unaffected reference individual. Thus the methods can include obtaining and analyzing a sample from a suitable reference individual.

Samples that are suitable for use in the methods described herein contain genetic material, e.g., genomic DNA (gDNA). Non-limiting examples of sources of samples include urine, blood, plasma, serum, saliva, semen, sputum, cerebral spinal fluid, tears, or mucus, or such a sample absorbed onto a paper or polymer substrate. A biological sample can be further fractionated, if desired, to a fraction containing particular cell types. For example, a blood sample can be fractionated into serum or into fractions containing particular types of blood cells such as red blood cells or white blood cells (leukocytes). If desired, a sample can be a combination of samples from a subject such as a combination of a tissue and fluid sample. The sample itself will typically consist of nucleated cells (e.g., blood or buccal cells), tissue, etc., removed from the subject. The subject can be an adult, child, fetus, or embryo. In some embodiments, the sample is obtained prenatally, either from a fetus or embryo or from the mother (e.g., from fetal or embryonic cells in the maternal circulation). Methods and reagents are known in the art for obtaining, processing, and analyzing samples. In some embodiments, the sample is obtained with the assistance of a health care provider, e.g., to draw blood. In some embodiments, the sample is obtained without the assistance of a health care provider, e.g., where the sample is obtained non-invasively, such as a sample comprising buccal cells that is obtained using a buccal swab or brush, or a saliva sample.

The sample may be processed before the detecting step. For example, DNA in a cell or tissue sample can be separated from other components of the sample. The sample can be concentrated and/or purified to isolate DNA. Cells can be harvested from a biological sample using standard techniques known in the art. For example, cells can be harvested by centrifuging a cell sample and resuspending the pelleted cells. The cells can be resuspended in a buffered solution such as phosphate-buffered saline (PBS). After centrifuging the cell suspension to obtain a cell pellet, the cells can be lysed to extract DNA, e.g., gDNA. See, e.g., Ausubel et al., 2003, supra. All samples obtained from a subject, including those subjected to any sort of further processing, are considered to be obtained from the subject.

The absence or presence of a haplotype associated with schizophrenia as described herein can be determined using methods known in the art, e.g., gel electrophoresis, capillary electrophoresis, size exclusion chromatography, sequencing, and/or arrays to detect the presence or absence of the marker(s) of the haplotype. Amplification of nucleic acids, where desirable, can be accomplished using methods known in the art, e.g., PCR.

As used herein, “detecting an allele or a haplotype,” “determining the presence of one or more alleles,” and “assaying for the presence of one or more alleles” includes obtaining information regarding the identity, presence or absence of one or more genetic markers in a subject. Determining or assaying for the presence of one or more alleles can, but need not, include obtaining a sample comprising DNA from a subject, and/or assessing the identity, presence or absence of one or more genetic markers in the sample. The individual or organization who detects, determines, or assays the allele or haplotype need not actually carry out the physical analysis of a sample from a subject; the information can be obtained by analysis of the sample by a third party. Thus the methods can include steps that occur at more than one site. For example, a sample can be obtained from a subject at a first site, such as at a health care provider, or at the subject's home in the case of a self-testing kit. The sample can be analyzed at the same or a second site, e.g., at a laboratory or other testing facility.

Detecting an allele or a haplotype and determining the presence of one or more alleles can also include or consist of reviewing a subject's medical history, where the medical history includes information regarding the identity, presence or absence of one or more genetic markers in the subject, e.g., results of a genetic test.

In some embodiments, to determine the presence of an allele or a haplotype described herein, a biological sample that includes nucleated cells (such as blood, a cheek swab, or saliva) is prepared and analyzed for the presence or absence of preselected markers. Such diagnoses may be performed by diagnostic laboratories. Alternatively, diagnostic kits containing probes or nucleic acid arrays useful in, e.g., determining the presence of one or more SNP alleles can be manufactured and sold to health care providers or to private individuals for self-diagnosis. Diagnostic or prognostic tests can be performed as described herein or using well known techniques, such as described in U.S. Pat. No. 5,800,998.

Results of these tests, and optionally interpretive information, can be returned to the subject, the health care provider, medical caregiver, physician, nurse, or to a third party payor. The results can be used in a number of ways. The information can be, e.g., communicated to the tested subject, e.g., with a prognosis and optionally interpretive materials that help the subject understand the test results and prognosis. The information can be used, e.g., by a health care provider, to determine whether to administer a specific drug, or whether a subject should be assigned to a specific category, e.g., a category associated with a specific disease phenotype, or with drug response or non-response. The information can be used, e.g., by a third party payor such as a healthcare payor (e.g., insurance company or HMO) or other agency, to determine whether or not to reimburse a health care provider for services to the subject, or whether to approve the provision of services to the subject. For example, the healthcare payor may decide to reimburse a health care provider for treatments for schizophrenia if the subject has an increased severity of negative symptoms of schizophrenia, e.g., a subject with three, four, five, or six risk alleles described in Table 1 or if the subject has three, four, five, or six folate alleles described in Table 1. As another example, a drug or treatment may be indicated for individuals with a certain haplotype, and the insurance company would only reimburse the health care provider (or the insured individual) for prescription or purchase of the drug if the insured individual has that haplotype. The presence or absence of the haplotype in a patient may be ascertained by using any of the methods described herein.

Information gleaned from the methods described herein can also be used to select or stratify subjects for a clinical trial. For example, the presence of a selected haplotype described herein can be used to select a subject for a trial. The information can optionally be correlated with clinical information about the subject, e.g., diagnostic or prognostic information.

### Linkage Disequilibrium Analysis

One of skill in the art will appreciate that markers within one Linkage Disequilibrium Unit (LDU) of the polymorphisms described herein can also be used in a similar manner to those described herein. Linkage disequilibrium (LD) is a measure of the degree of association between alleles in a population. LDUs share an inverse relationship with LD so that regions with high LD (such as haplotype blocks) have few LDUs and low recombination, while regions with many LDUs have low LD and high recombination. Methods of calculating LDUs are known in the art (see, e.g., Morton et al., Proc Natl Acad Sci USA 98(9):5217-21 (2001); Tapper et al., Proc Natl Acad Sci USA 102(33):11835-11839 (2005); Maniatis et al., Proc Natl Acad Sci USA 99:2228-2233 (2002)). Thus, in some embodiments, the methods include analysis of polymorphisms that are within one LDU of a polymorphism described herein.

Alternatively, methods described herein can include analysis of polymorphisms that are within a value defined by Lewontin's D′ (linkage disequilibrium parameter, see Lewontin, Genetics 49:49-67 (1964)) of a polymorphism described herein. Results can be obtained, e.g., from on line public resources such as HapMap.org. The simple linkage disequilibrium parameter (D) reflects the degree to which alleles at two loci (for example two SNPs) occur together more often (positive values) or less often (negative values) than expected in a population as determined by the products of their respective allele frequencies. For any two loci, D can vary in value from −0.25 to +0.25. However, the magnitude of D (Dmax) varies as function of allele frequencies. To control for this, Lewontin introduced the D′ parameter, which is D/Dmax and varies in value from −1 (alleles never observed together) to +1 (alleles always observed together). Typically, the absolute value of D′ (i.e., |D′|) is reported in online databases, because it follows mathematically that positive association for one set of alleles at two loci corresponds to a negative association of equal magnitude for the reciprocal set. This disequilibrium parameter varies from 0 (no association of alleles at the two loci) to 1 (maximal possible association of alleles at the two loci).

Thus, in some embodiments, the methods include analysis of polymorphisms that are in complete linkage disequilibrium, i.e., with an R²=1 or a D′=1, for pairwise comparisons, of a polymorphism described herein.

Methods are known in the art for identifying suitable polymorphisms; for example, the International HapMap Project provides a public database that can be used, see, hapmap.org, as well as The International HapMap Consortium, Nature 426:789-796 (2003), and The International HapMap Consortium, Nature 437:1299-1320 (2005). Generally, it will be desirable to use a HapMap constructed using data from individuals who share ethnicity with the subject, e.g., a HapMap for Caucasians would ideally be used to identify markers within one LDU or with an R²=1 or D′=1 of a marker described herein for use in genotyping a subject of Caucasian descent.

### Identification of Additional Markers for Use in the Methods Described Herein

Skilled practitioners will also appreciate that additional markers can be used.

In general, genetic markers can be identified using any of a number of methods well known in the art. For example, numerous polymorphisms in the regions described herein are known to exist and are available in public databases, which can be searched using methods and algorithms known in the art. Alternately, polymorphisms can be identified by sequencing either genomic DNA or cDNA in the region in which it is desired to find a polymorphism. According to one approach, primers are designed to amplify such a region, and DNA from a subject is obtained and amplified. The DNA is sequenced, and the sequence (referred to as a “subject sequence” or “test sequence”) is compared with a reference sequence, which can represent the “normal” or “wild type” sequence, or the “affected” sequence. In some embodiments, a reference sequence can be from, for example, the human draft genome sequence, publicly available in various databases, or a sequence deposited in a database such as GenBank. In some embodiments, the reference sequence is a composite of ethnically diverse individuals.

In general, if sequencing reveals a difference between the sequenced region and the reference sequence, a polymorphism has been identified. The fact that a difference in nucleotide sequence is identified at a particular site determines that a polymorphism exists at that site. In most instances, particularly in the case of SNPs, only two polymorphic variants will exist at any location. However, in the case of SNPs, up to four variants may exist since there are four naturally occurring nucleotides in DNA. Other polymorphisms, such as insertions and deletions, may have more than four alleles.

Methods of nucleic acid analysis to assay for polymorphisms and/or polymorphic variants include, e.g., microarray analysis. Hybridization methods, such as Southern analysis, Northern analysis, or in situ hybridizations, can also be used (see Current Protocols in Molecular Biology, Ausubel et al., Eds., John Wiley & Sons, 2003). To assay for microdeletions, fluorescence in situ hybridization (FISH) using DNA probes that are directed to a putatively deleted region in a chromosome can be used. For example, probes that detect all or a part of a microsatellite marker can be used to detect microdeletions in the region that contains that marker.

Other methods include direct manual sequencing (Church and Gilbert, Proc. Natl. Acad. Sci. USA 81:1991-1995 (1988); Sanger et al., Proc. Natl. Acad. Sci. 74:5463-5467 (1977); Beavis et al., U.S. Pat. No. 5,288,644); automated fluorescent sequencing; single-stranded conformation polymorphism assays (SSCP); clamped denaturing gel electrophoresis (CDGE); two-dimensional gel electrophoresis (2DGE or TDGE); conformational sensitive gel electrophoresis (CSGE); denaturing gradient gel electrophoresis (DGGE) (Sheffield et al., Proc. Natl. Acad. Sci. USA 86:232-236 (1989)), mobility shift analysis (Orita et al., Proc. Natl. Acad. Sci. USA 86:2766-2770 (1989)), restriction enzyme analysis (Flavell et al., Cell 15:25 (1978); Geever et al., Proc. Natl. Acad. Sci. USA 78:5081 (1981)); quantitative real-time PCR (Raca et al., Genet Test 8(4):387-94 (2004)); heteroduplex analysis; chemical mismatch cleavage (CMC) (Cotton et al., Proc. Natl. Acad. Sci. USA 85:4397-4401 (1985)); RNase protection assays (Myers et al., Science 230:1242 (1985)); use of polypeptides that recognize nucleotide mismatches, e.g., E. coli mutS protein; allele-specific PCR, for example. See, e.g., Gerber et al., U.S. Patent Publication No. 2004/0014095, which is incorporated herein by reference in its entirety. In some embodiments, the sequence is determined on both strands of DNA.

To assay for polymorphisms and/or polymorphic variants, it will frequently be desirable to amplify a portion of genomic DNA (gDNA) encompassing the polymorphic site. Such regions can be amplified and isolated by PCR using oligonucleotide primers designed based on genomic and/or cDNA sequences that flank the site. See, e.g., PCR Primer: A Laboratory Manual, Dieffenbach and Dveksler, (Eds.); McPherson et al., PCR Basics: From Background to Bench (Springer Verlag, 2000); Mattila et al., Nucleic Acids Res., 19:4967 (1991); Eckert et al., PCR Methods and Applications, 1:17 (1991); PCR (Eds. McPherson et al., IRL Press, Oxford); and U.S. Pat. No. 4,683,202. Other amplification methods that may be employed include the ligase chain reaction (LCR) (Wu and Wallace, Genomics, 4:560 (1989), Landegren et al., Science, 241:1077 (1988), transcription amplification (Kwoh et al., Proc. Natl. Acad. Sci. USA, 86:1173 (1989)), self-sustained sequence replication (Guatelli et al., Proc. Nat. Acad. Sci. USA, 87:1874 (1990)), and nucleic acid based sequence amplification (NASBA). Guidelines for selecting primers for PCR amplification are well known in the art. See, e.g., McPherson et al., PCR Basics: From Background to Bench, Springer-Verlag, 2000. A variety of computer programs for designing primers are available, e.g., ‘Oligo’ (National Biosciences, Inc, Plymouth Minn.), MacVector (Kodak/IBI), and the GCG suite of sequence analysis programs (Genetics Computer Group, Madison, Wis.).

In one example, a sample (e.g., a sample comprising genomic DNA), is obtained from a subject. The DNA in the sample is then examined to assay for an allele or a haplotype as described herein. The allele or haplotype can be detected by any method described herein, e.g., by sequencing or by hybridization of the gene in the genomic DNA, RNA, or cDNA to a nucleic acid probe, e.g., a DNA probe (which includes cDNA and oligonucleotide probes) or an RNA probe. The nucleic acid probe can be designed to specifically or preferentially hybridize with a particular polymorphic variant.

In some embodiments, a peptide nucleic acid (PNA) probe can be used instead of a nucleic acid probe in the hybridization methods described above. PNA is a DNA mimetic with a peptide-like, inorganic backbone, e.g., N-(2-aminoethyl)glycine units, with an organic base (A, G, C, T, or U) attached to the glycine nitrogen via a methylene carbonyl linker (see, e.g., Nielsen et al., Bioconjugate Chemistry, The American Chemical Society, 5:1 (1994)). The PNA probe can be designed to specifically hybridize to a nucleic acid comprising a polymorphic variant conferring increased severity of negative symptoms of schizophrenia or treatment response to folate and/or vitamin B12.

In some embodiments, restriction digest analysis can be used to assay for the existence of a polymorphic variant of a polymorphism, if alternate polymorphic variants of the polymorphism result in the creation or elimination of a restriction site. A sample containing genomic DNA is obtained from the individual. Polymerase chain reaction (PCR) can be used to amplify a region comprising the polymorphic site, and restriction fragment length polymorphism analysis is conducted (see Ausubel et al., Current Protocols in Molecular Biology, supra). The digestion pattern of the relevant DNA fragment indicates the presence or absence of a particular polymorphic variant of the polymorphism and is therefore indicative of an increase or decrease in severity of negative symptoms of schizophrenia or treatment response to folate and/or vitamin B12.

Sequence analysis can also be used to detect specific polymorphic variants. A sample comprising DNA or RNA is obtained from the subject. PCR or other appropriate methods can be used to amplify a portion encompassing the polymorphic site, if desired. The sequence is then ascertained, using any standard method, and the presence of a polymorphic variant is determined.

Allele-specific oligonucleotides can also be used to assay for the presence of a polymorphic variant, e.g., through the use of dot-blot hybridization of amplified oligonucleotides with allele-specific oligonucleotide (ASO) probes (see, for example, Saiki et al., Nature (London) 324:163-166 (1986)). An “allele-specific oligonucleotide” (also referred to herein as an “allele-specific oligonucleotide probe”) is typically an oligonucleotide of approximately 10-50 base pairs, preferably approximately 15-30 base pairs, that specifically hybridizes to a nucleic acid region that contains a polymorphism. An allele-specific oligonucleotide probe that is specific for a particular polymorphism can be prepared using standard methods (see Ausubel et al., Current Protocols in Molecular Biology, supra).

Generally, to determine which of multiple polymorphic variants is present in a subject, a sample comprising DNA is obtained from the individual. PCR can be used to amplify a portion encompassing the polymorphic site. DNA containing the amplified portion may be dot-blotted, using standard methods (see Ausubel et al., Current Protocols in Molecular Biology, supra), and the blot contacted with the oligonucleotide probe. The presence of specific hybridization of the probe to the DNA is then detected. Specific hybridization of an allele-specific oligonucleotide probe (specific for a polymorphic variant indicative of increased severity of negative symptoms of schizophrenia or treatment response to folate and/or vitamin B12) to DNA from the subject is indicative of increased severity of negative symptoms of schizophrenia or treatment response to folate and/or vitamin B12.

In some embodiments, fluorescence polarization template-directed dye-terminator incorporation (FP-TDI) is used to determine which of multiple polymorphic variants of a polymorphism is present in a subject (Chen et al., Genome Research 9(5):492-498 (1999)). Rather than involving use of allele-specific probes or primers, this method employs primers that terminate adjacent to a polymorphic site, so that extension of the primer by a single nucleotide results in incorporation of a nucleotide complementary to the polymorphic variant at the polymorphic site.

Real-time pyrophosphate DNA sequencing is yet another approach to detection of polymorphisms and polymorphic variants (Alderborn et al., (2000) Genome Research, 10(8):1249-1258). Additional methods include, for example, PCR amplification in combination with denaturing high performance liquid chromatography (dHPLC) (Underhill, P. A., et al., Genome Research, Vol. 7, No. 10, pp. 996-1005, 1997).

The methods can include determining the genotype of a subject with respect to both copies of the polymorphic site present in the genome. For example, the complete genotype may be characterized as −/−, as −/+, or as +/+, where a minus sign indicates the presence of the reference or wild type sequence at the polymorphic site, and the plus sign indicates the presence of a polymorphic variant other than the reference sequence. If multiple polymorphic variants exist at a site, this can be appropriately indicated by specifying which ones are present in the subject. Any of the detection means described herein can be used to determine the genotype of a subject with respect to one or both copies of the polymorphism present in the subject's genome.

In some embodiments, it is desirable to employ methods that can detect the presence of multiple polymorphisms (e.g., polymorphic variants at a plurality of polymorphic sites) in parallel or substantially simultaneously. Oligonucleotide arrays represent one suitable means for doing so. Other methods, including methods in which reactions (e.g., amplification, hybridization) are performed in individual vessels, e.g., within individual wells of a multi-well plate or other vessel may also be performed so as to detect the presence of multiple polymorphic variants (e.g., polymorphic variants at a plurality of polymorphic sites) in parallel or substantially simultaneously according to certain embodiments of the invention.

### Probes

Nucleic acid probes can be used to detect and/or quantify the presence of a particular target nucleic acid sequence within a sample of nucleic acid sequences, e.g., as hybridization probes, or to amplify a particular target sequence within a sample, e.g., as a primer. Probes have a complimentary nucleic acid sequence that selectively hybridizes to the target nucleic acid sequence. In order for a probe to hybridize to a target sequence, the hybridization probe must have sufficient identity with the target sequence, i.e., at least 70%, e.g., 80%, 90%, 95%, 98% or more identity to the target sequence. The probe sequence must also be sufficiently long so that the probe exhibits selectivity for the target sequence over non-target sequences. For example, the probe will be at least 20, e.g., 25, 30, 35, 50, 100, 200, 300, 400, 500, 600, 700, 800, 900 or more, nucleotides in length. In some embodiments, the probes are not more than 30, 50, 100, 200, 300, 500, 750, or 1000 nucleotides in length. Probes are typically about 20 to about 1×10⁶ nucleotides in length. Probes include primers, which generally refers to a single-stranded oligonucleotide probe that can act as a point of initiation of template-directed DNA synthesis using methods such as PCR (polymerase chain reaction), LCR (ligase chain reaction), etc., for amplification of a target sequence. In some embodiments, the probe is a test probe, e.g., a probe that can be used to detect polymorphisms in a region described herein, e.g., polymorphisms as described herein. In some embodiments, the probe can bind to another marker sequence associated with schizophrenia, as described herein.

Control probes can also be used. For example, a probe that binds a less variable sequence, e.g., repetitive DNA associated with a centromere of a chromosome, can be used as a control. Probes that hybridize with various centromeric DNA and locus-specific DNA are available commercially, for example, from Vysis, Inc. (Downers Grove, Ill.), Molecular Probes, Inc. (Eugene, Oreg.), or from Cytocell (Oxfordshire, UK). Probe sets are available commercially, e.g., from Applied Biosystems, e.g., the Assays-on-Demand SNP kits. Alternatively, probes can be synthesized, e.g., chemically or in vitro, or made from chromosomal or genomic DNA through standard techniques. For example, sources of DNA that can be used include genomic DNA, cloned DNA sequences, somatic cell hybrids that contain one, or a part of one, human chromosome along with the normal chromosome complement of the host, and chromosomes purified by flow cytometry or microdissection. The region of interest can be isolated through cloning, or by site-specific amplification via the polymerase chain reaction (PCR). See, e.g., Nath and Johnson, Biotechnic. Histochem., 1998, 73(1):6-22, Wheeless et al., Cytometry 1994, 17:319-326, and U.S. Pat. No. 5,491,224.

In some embodiments, the probes are labeled, e.g., by direct labeling, with a fluorophore, an organic molecule that fluoresces after absorbing light of lower wavelength/higher energy. A directly labeled fluorophore allows the probe to be visualized without a secondary detection molecule. After covalently attaching a fluorophore to a nucleotide, the nucleotide can be directly incorporated into the probe with standard techniques such as nick translation, random priming, and PCR labeling. Alternatively, deoxycytidine nucleotides within the probe can be transaminated with a linker. The fluorophore then is covalently attached to the transaminated deoxycytidine nucleotides. See, e.g., U.S. Pat. No. 5,491,224.

Fluorophores of different colors can be chosen such that each probe in a set can be distinctly visualized. For example, a combination of the following fluorophores can be used: 7-amino-4-methylcoumarin-3-acetic acid (AMCA), TEXAS RED™ (Molecular Probes, Inc., Eugene, Oreg.), 5-(and-6)-carboxy-X-rhodamine, lissamine rhodamine B, 5-(and-6)-carboxyfluorescein, fluorescein-5-isothiocyanate (FITC), 7-diethylaminocoumarin-3-carboxylic acid, tetramethylrhodamine-5-(and-6)-isothiocyanate, 5-(and-6)-carboxytetramethylrhodamine, 7-hydroxycoumarin-3-carboxylic acid, 6-[fluorescein 5-(and-6)-carboxamido]hexanoic acid, N-(4,4-difluoro-5,7-dimethyl-4-bora-3a,4a diaza-3-indacenepropionic acid, eosin-5-isothiocyanate, erythrosin-5-isothiocyanate, and CASCADE™ blue acetylazide (Molecular Probes, Inc., Eugene, Oreg.). Fluorescently labeled probes can be viewed with a fluorescence microscope and an appropriate filter for each fluorophore, or by using dual or triple band-pass filter sets to observe multiple fluorophores. See, for example, U.S. Pat. No. 5,776,688. Alternatively, techniques such as flow cytometry can be used to examine the hybridization pattern of the probes. Fluorescence-based arrays are also known in the art.

In other embodiments, the probes can be indirectly labeled with, e.g., biotin or digoxygenin, or labeled with radioactive isotopes such as ³²P and ³H. For example, a probe indirectly labeled with biotin can be detected by avidin conjugated to a detectable marker. For example, avidin can be conjugated to an enzymatic marker such as alkaline phosphatase or horseradish peroxidase. Enzymatic markers can be detected in standard colorimetric reactions using a substrate and/or a catalyst for the enzyme. Catalysts for alkaline phosphatase include 5-bromo-4-chloro-3-indolylphosphate and nitro blue tetrazolium. Diaminobenzoate can be used as a catalyst for horseradish peroxidase.

Oligonucleotide probes that exhibit differential or selective binding to polymorphic sites may readily be designed by one of ordinary skill in the art. For example, an oligonucleotide that is perfectly complementary to a sequence that encompasses a polymorphic site (i.e., a sequence that includes the polymorphic site, within it or at one end) will generally hybridize preferentially to a nucleic acid comprising that sequence, as opposed to a nucleic acid comprising an alternate polymorphic variant.

### Arrays and Uses Thereof

Arrays that include a substrate having a plurality of addressable areas and methods of using them are also provided. At least one area of the plurality includes a nucleic acid probe that binds specifically to a sequence comprising a polymorphism listed in Table 1, and can be used to detect the absence or presence of said polymorphism, e.g., one or more SNPs, microsatellites, minisatellites, or indels, as described herein, to determine a haplotype. For example, the array can include one or more nucleic acid probes that can be used to detect a polymorphism listed in Table 1. In some embodiments, the array further includes at least one area that includes a nucleic acid probe that can be used to specifically detect another marker associated with schizophrenia, as described herein. The substrate can be, e.g., a two-dimensional substrate known in the art such as a glass slide, a wafer (e.g., silica or plastic), a mass spectroscopy plate, or a three-dimensional substrate such as a gel pad. In some embodiments, the probes are nucleic acid capture probes.

Methods for generating arrays are known in the art and include, e.g., photolithographic methods (see, e.g., U.S. Pat. Nos. 5,143,854; 5,510,270; and 5,527,681), mechanical methods (e.g., directed-flow methods as described in U.S. Pat. No. 5,384,261), pin-based methods (e.g., as described in U.S. Pat. No. 5,288,514), and bead-based techniques (e.g., as described in PCT US/93/04145). The array typically includes oligonucleotide probes capable of specifically hybridizing to different polymorphic variants. According to the method, a nucleic acid of interest, e.g., a nucleic acid encompassing a polymorphic site, (which is typically amplified) is hybridized with the array and scanned. Hybridization and scanning are generally carried out according to standard methods. See, e.g., WO 92/10092 and WO 95/11995, and U.S. Pat. No. 5,424,186. After hybridization and washing, the array is scanned to determine the position on the array to which the nucleic acid hybridizes. The hybridization data obtained from the scan is typically in the form of fluorescence intensities as a function of location on the array.

Arrays can include multiple detection blocks (i.e., multiple groups of probes designed for detection of particular polymorphisms). Such arrays can be used to analyze multiple different polymorphisms. Detection blocks may be grouped within a single array or in multiple, separate arrays so that varying conditions (e.g., conditions optimized for particular polymorphisms) may be used during the hybridization. For example, it may be desirable to provide for the detection of those polymorphisms that fall within G-C rich stretches of a genomic sequence, separately from those falling in A-T rich segments.

Additional description of use of oligonucleotide arrays for detection of polymorphisms can be found, for example, in U.S. Pat. Nos. 5,858,659 and 5,837,832. In addition to oligonucleotide arrays, cDNA arrays may be used similarly in certain embodiments of the invention.

The methods described herein can include providing an array as described herein; contacting the array with a sample, e.g., a portion of genomic DNA that includes at least one marker described herein or another chromosome, e.g., including another region or marker associated with schizophrenia, and detecting binding of a nucleic acid from the sample to the array. Optionally, the method includes amplifying nucleic acid from the sample, e.g., genomic DNA that includes a portion of a human chromosome described herein, and, optionally, a region that includes another region associated with schizophrenia, prior to or during contact with the array.

In some aspects, the methods described herein can include using an array that can ascertain differential expression patterns or copy numbers of one or more genes in samples from normal and affected individuals (see, e.g., Redon et al., Nature. 444(7118):444-54 (2006)). For example, arrays of probes to a marker described herein can be used to measure polymorphisms between DNA from a subject having schizophrenia, and control DNA, e.g., DNA obtained from an individual who does not have schizophrenia, and has no risk factors for schizophrenia. Since the clones on the array contain sequence tags, their positions on the array are accurately known relative to the genomic sequence. Different hybridization patterns between DNA from an individual afflicted with schizophrenia and DNA from a normal individual at areas in the array corresponding to markers as described herein, and, optionally, one or more other regions associated with schizophrenia, are indicative of an increased severity of negative symptoms of schizophrenia or treatment response to folate and/or vitamin B12. Methods for array production, hybridization, and analysis are described, e.g., in Snijders et al., (2001) Nat. Genetics 29:263-264; Klein et al., (1999) Proc. Natl. Acad. Sci. U.S.A. 96:4494-4499; Albertson et al., (2003) Breast Cancer Research and Treatment 78:289-298; and Snijders et al. “BAC microarray based comparative genomic hybridization.” In: Zhao et al. (Eds.), Bacterial Artificial Chromosomes: Methods and Protocols, Methods in Molecular Biology, Humana Press, 2002. Real time quantitative PCR can also be used to determine copy number.

In another aspect, the invention features methods of determining the absence or presence of an allele or a haplotype associated with schizophrenia as described herein, using an array described above. The methods include providing a two dimensional array having a plurality of addresses, each address of the plurality being positionally distinguishable from each other address of the plurality having a unique nucleic acid capture probe, contacting the array with a first sample from a test subject who is suspected of having or being at risk for schizophrenia, and comparing the binding of the first sample with one or more references, e.g., binding of a sample from a subject who is known to have schizophrenia, and/or binding of a sample from a subject who is unaffected, e.g., a control sample from a subject who neither has, nor has any risk factors for schizophrenia. In some embodiments, the methods include contacting the array with a second sample from a subject who has schizophrenia; and comparing the binding of the first sample with the binding of the second sample. In some embodiments, the methods include contacting the array with a third sample from a cell or subject that does not have schizophrenia and is not at risk for schizophrenia; and comparing the binding of the first sample with the binding of the third sample. In some embodiments, the second and third samples are from first or second-degree relatives of the test subject. Binding, e.g., in the case of a nucleic acid hybridization, with a capture probe at an address of the plurality, can be detected by any method known in the art, e.g., by detection of a signal generated from a label attached to the nucleic acid.

### Kits

Also within the scope of the invention are kits comprising a probe that hybridizes with a region of human chromosome as described herein and can be used to detect a polymorphism described herein. The kit can include one or more other elements including: instructions for use; and other reagents, e.g., a label, or an agent useful for attaching a label to the probe. Instructions for use can include instructions for diagnostic applications of the probe for predicting response to treatment of negative symptoms of schizophrenia in a method described herein. Other instructions can include instructions for attaching a label to the probe, instructions for performing in situ analysis with the probe, and/or instructions for obtaining a sample to be analyzed from a subject. As discussed above, the kit can include a label, e.g., any of the labels described herein. In some embodiments, the kit includes a labeled probe that hybridizes to a region of human chromosome as described herein, e.g., a labeled probe as described herein.

The kit can also include one or more additional probes that hybridize to the same chromosome or another chromosome or portion thereof that can have an abnormality associated with severity of negative symptoms. A kit that includes additional probes can further include labels, e.g., one or more of the same or different labels for the probes. In other embodiments, the additional probe or probes provided with the kit can be a labeled probe or probes. When the kit further includes one or more additional probe or probes, the kit can further provide instructions for the use of the additional probe or probes.

Kits for use in self-testing can also be provided. For example, such test kits can include devices and instructions that a subject can use to obtain a sample, e.g., of buccal cells or blood, without the aid of a health care provider. For example, buccal cells can be obtained using a buccal swab or brush, or using mouthwash.

Kits as provided herein can also include a mailer, e.g., a postage paid envelope or mailing pack, that can be used to return the sample for analysis, e.g., to a laboratory. The kit can include one or more containers for the sample, or the sample can be in a standard blood collection vial. The kit can also include one or more of an informed consent form, a test requisition form, and instructions on how to use the kit in a method described herein. Methods for using such kits are also included herein. One or more of the forms, e.g., the test requisition form, and the container holding the sample, can be coded, e.g., with a bar code, for identifying the subject who provided the sample.

In some embodiments, the kits can include one or more reagents for processing a biological sample. For example, a kit can include reagents for isolating mRNA or genomic DNA from a biological sample and/or reagents for amplifying isolated mRNA (e.g., reverse transcriptase, primers for reverse transcription or PCR amplification, or dNTPs) and/or genomic DNA. The kits can also, optionally, contain one or more reagents for detectably-labeling an mRNA, mRNA amplicon, genomic DNA or DNA amplicon, which reagents can include, e.g., an enzyme such as a Klenow fragment of DNA polymerase, T4 polynucleotide kinase, one or more detectably-labeled dNTPs, or detectably-labeled gamma phosphate ATP (e.g., ³³P-ATP).

In some embodiments, the kits can include a software package for analyzing the results of, e.g., a microarray analysis or expression profile.

### Databases

Also provided herein are databases that include a list of polymorphisms as described herein, and wherein the list is largely or entirely limited to polymorphisms identified as useful in performing genetic diagnosis of or determination of severity of negative symptoms of schizophrenia. The list is stored, e.g., on a flat file or computer-readable medium. The databases can further include information regarding one or more subjects, e.g., whether a subject is affected or unaffected, clinical information such as age of onset of symptoms, any treatments administered and outcomes (e.g., data relevant to pharmacogenomics, diagnostics, or theranostics), and other details, e.g., about the disorder in the subject, or environmental or other genetic factors. The databases can be used to detect correlations between a particular haplotype and the information regarding the subject, e.g., to detect correlations between a haplotype and a particular phenotype, or treatment response.

### Engineered Cells

Also provided herein are engineered cells that harbor one or more polymorphism described herein, e.g., three, four, five, or six polymorphisms that constitute a haplotype associated with severity of negative symptoms of schizophrenia or treatment response to folate and/or vitamin B12. Such cells are useful for studying the effect of one or more polymorphism on physiological function, and for identifying and/or evaluating potential therapeutic agents for the treatment of negative symptoms of schizophrenia, e.g., folate and vitamin B12.

As one example, included herein are cells in which one of the various alleles of the genes described herein has been re-created that are associated with an increased severity of negative symptoms or decrease in negative symptoms in response to folate and/or vitamin B12 treatment. Methods are known in the art for generating cells, e.g., by homologous recombination between the endogenous gene and an exogenous DNA molecule introduced into a cell, e.g., a cell of an animal. In some embodiments, the cells can be used to generate transgenic animals using methods known in the art.

The cells are preferably mammalian cells, e.g., epithelial or endothelial type cells, in which an endogenous gene has been altered to include a polymorphism as described herein. Techniques such as targeted homologous recombinations can be used to insert the heterologous DNA as described in, e.g., Chappel, U.S. Pat. No. 5,272,071; WO 91/06667, published in May 16, 1991.

### Subjects to be Treated

A subject can be selected on the basis that they have, or are at risk of developing, schizophrenia. It is well within the skills of an ordinary practitioner to recognize a subject that has, or is at risk of developing, schizophrenia. A subject that has, or is at risk of developing, schizophrenia is one having one or more symptoms of the condition or one or more risk factors for developing the condition. Symptoms of schizophrenia are known to those of skill in the art and include, without limitation, loss of interest in everyday activities, appearing to lack emotion, reduced ability to plan or carry out activities, neglect of personal hygiene, social withdrawal, loss of motivation, delusions, hallucinations, thought disorder, problems with making sense of information, difficulty paying attention, memory problems, disorganized behavior, depression, and mood swings. A subject that has, or is at risk of developing, schizophrenia is one with known risk factors such as complications during pregnancy or birth (e.g., a child who experiences oxygen deprivation during pregnancy, bleeding during pregnancy, maternal malnutrition, infections during pregnancy, long labor, prematurity, and low birth weight), stress, poor nutrition, and certain family backgrounds.

The methods are effective for a variety of subjects including mammals, e.g., humans and other animals, such as laboratory animals, e.g., mice, rats, rabbits, or monkeys, or domesticated and farm animals, e.g., cats, dogs, goats, sheep, pigs, cows, or horses.

### Folate

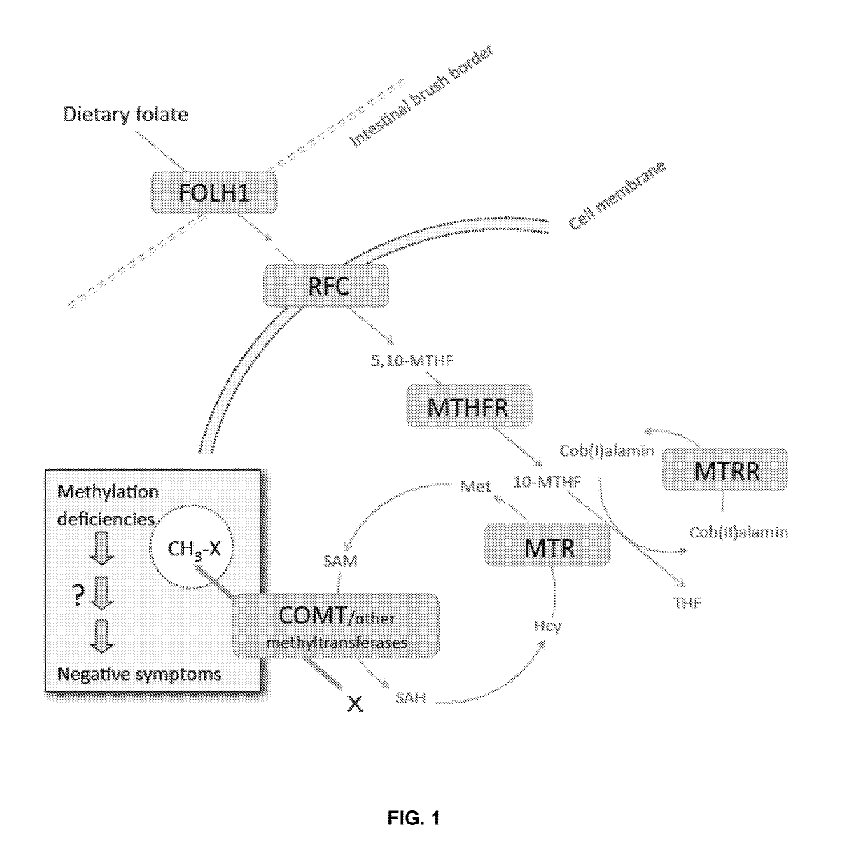

Folate supplies the substrate for intracellular methylation reactions that are essential to normal brain development and function. Methylation governs such vital processes as DNA synthesis and repair, gene expression, neurotransmitter synthesis and degradation, and homocysteine metabolism (Frankenburg, Harv Rev Psychiatry 15:146-160, 2007). The availability of one-carbon moieties for methylation reactions is regulated both by dietary folate intake and by cellular machinery mediating folate absorption through the gut, translocation of folate into cells, and conversion of precursors to methyl donors such as S-adenosylmethionine (SAM; FIG. 1) (Greene et al., Hum Mol Genet 18:R113-129, 2009).

Folate, also known as folic acid, as well as pteroyl-L-glutamic acid, is essential to numerous bodily functions ranging from nucleotide biosynthesis to the remethylation of homocysteine. The human body needs folate to synthesize DNA, repair DNA, and methylate DNA as well as to act as a cofactor in biological reactions involving folate. It is especially important during periods of rapid cell division and growth. A lack of dietary folic acid leads to folate deficiency. This can result in many health problems, the most notable one being neural tube defects in developing embryos. Low levels of folate can also lead to homocysteine accumulation as a result of the impairment of one-carbon metabolism mechanism methylation. The exact mechanisms involved in the development of schizophrenia are not entirely clear but may have something to do with DNA methylation and one carbon metabolism; these are the precise roles of folate in the body.

### Vitamin B12

Vitamin B12, also known as cobalamin, is a water soluble vitamin with a key role in normal functioning of the brain and nervous system, and for the formation of blood. It is normally involved in cellular metabolism, especially affecting DNA synthesis and regulation, but also fatty acid synthesis and energy production. Many of the functions of vitamin B12 can be replaced by sufficient quantities of folic acid, however, vitamin B12 is required to regenerate folate in the body. Most vitamin B12 deficiency symptoms are actually folate deficiency symptoms, since they include all the effects of pernicious anemia and megaloblastosis, which are due to poor DNA synthesis when the body does not have a proper supply of folic acid for the production of thymine. When sufficient folic acid is available, all known vitamin B12 related deficiency syndromes normalize, except those connected with the vitamin B12-dependent enzymes such as MTR and the buildup of its substrate, homocysteine.

In all of the methods described herein, appropriate dosages of folate and derivatives thereof, e.g, folic acid, DEPLIN® (L-methylfolate), 5-formyltetrahyrofolate, 10-formyltetrahyrofolate, and vitamin B12 can readily be determined by those of ordinary skill in the art of medicine by monitoring the patient for signs of disease amelioration or inhibition, and increasing or decreasing the dosage and/or frequency of treatment as desired. For example, folate dosage can range from 0.1 milligram to 1000 milligrams per day, e.g., 0.1, 0.2, 0.3, 0.4, 0.5, 0.6, 0.7, 0.8, 0.9, 1, 2, 3, 4, 5, 10, 50, 100, 200, 300, 400, 500, 600, and 800 milligrams per day. Vitamin B12 dosage can range from 1 microgram to 20 micrograms per day, e.g., 2, 3, 4, 5, 6, 7, 8, 9, 10, 12, 15, and 18 micrograms per day, and can be provided as cobalamin, cyanocobalamin, hydroxocobalamin (a form produced by bacteria), methylcobalamin, and adenosylcobalamin.

The pharmaceutical compositions can be administered to the patient by any, or a combination, of several routes, such as oral, intravenous, trans-mucosal (e.g., nasal, vaginal, etc.), pulmonary, transdermal, ocular, buccal, sublingual, intraperitoneal, intrathecal, intramuscular, parenteral, or long term depot preparation. Solid compositions for oral administration can contain suitable carriers or excipients, such as corn starch, gelatin, lactose, acacia, sucrose, microcrystalline cellulose, kaolin, mannitol, dicalcium phosphate, calcium carbonate, sodium chloride, lipids, alginic acid, or ingredients for controlled slow release. Disintegrators that can be used include, without limitation, micro-crystalline cellulose, corn starch, sodium starch glycolate and alginic acid. Tablet binders that may be used include, without limitation, acacia, methylcellulose, sodium carboxymethylcellulose, polyvinylpyrrolidone (Povidone), hydroxypropyl methylcellulose, sucrose, starch, and ethylcellulose.

Liquid compositions for oral administration prepared in water or other aqueous vehicles can include solutions, emulsions, syrups, and elixirs containing, together with the active compound(s), wetting agents, sweeteners, coloring agents, and flavoring agents. Various liquid and powder compositions can be prepared by conventional methods for inhalation into the lungs of the patient to be treated.

Injectable compositions may contain various carriers such as vegetable oils, dimethylacetamide, dimethylformamide, ethyl lactate, ethyl carbonate, isopropyl myristate, ethanol, and polyols (glycerol, propylene glycol, liquid polyethylene glycol, and the like). For intravenous injections, the compounds may be administered by the drip method, whereby a pharmaceutical composition containing the active compound(s) and a physiologically acceptable excipient is infused. Physiologically acceptable excipients may include, for example, 5% dextrose, 0.9% saline, Ringer's solution or other suitable excipients. For intramuscular preparations, a sterile composition of a suitable soluble salt form of the compound can be dissolved and administered in a pharmaceutical excipient such as Water-for-Injection, 0.9% saline, or 5% glucose solution, or depot forms of the compounds (e.g., decanoate, palmitate, undecylenic, enanthate) can be dissolved in sesame oil. Alternatively, the pharmaceutical composition can be formulated as a chewing gum, lollipop, or the like.

The subjects can also be those undergoing any of a variety of schizophrenia treatments. Thus, for example, subjects can be those being treated with one or more antipsychotic agents, selective serotonin reuptake inhibitors (SSRIs), glutamatergic compounds, estrogen, clozapine, N-methyl-D-aspartic acid agonists (e.g., glycine and D-serine), D-cycloserine, acetylcholinesterase inhibitors (e.g., galantamine, rivastigmine, and donepezil), folate, and vitamin B12.

The invention is further described in the following examples, which do not limit the scope of the invention described in the claims.

### EXAMPLES

The invention is further described in the following examples, which do not limit the scope of the invention described in the claims.

Altered folate metabolism has been implicated in several neuropsychiatric disorders, including schizophrenia. Reduced maternal folate intake (St Clair et al., JAMA 294:557-562, 2005) and increased maternal homocysteine blood level (Brown et al., Arch Gen Psychiatry 64:31-39, 2007) during neurodevelopment have been associated with substantial increases in schizophrenia risk. Low blood levels of folate have been observed in several cohorts of schizophrenia patients (Goff et al., Am J Psychiatry 161:1705-1708, 2004; Herran et al., Psychiatry Clin Neurosci 53:531-533, 1999; Muntjewerff et al., Psychiatry Res 121:1-9, 2003), and vitamin supplementation regimens that include folate (Levine et al., Biol Psychiatry 60:265-269, 2006) and methylfolate (Godfrey et al., Lancet 336:392-395, 1990) have been associated with symptomatic improvement. Moreover, genetic variants in two genes regulating folate metabolism, MTHFR (Allen et al., the SzGene database. Nat Genet 40:827-834, 2008) and MTR (Kempisty et al., Psychiatr Genet 17:177-181, 2007), have been associated with increased schizophrenia risk. MTHFR in particular has emerged as a strong candidate gene, with the low-functioning 677T allele significantly augmenting schizophrenia risk across 20 case-control studies (Allen et al., the SzGene database. Nat Genet 40:827-834, 2008), although not reaching the threshold of genome-wide significance.

One specific aspect of schizophrenia, negative symptoms, exhibits especially strong ties to folate metabolism and to folate-related genes. Negative symptoms, which include apathy, impoverished speech, flattened affect, and social withdrawal, contribute greatly to functional disability in schizophrenia and are not substantially improved by antipsychotic medications (Goff et al., Schizophrenia. Med Clin North Am 85:663-689, 2001; Lieberman et al., N Engl J Med 353:1209-1223, 2005; Mohamed et al., Am J Psychiatry 165:978-987, 2008). Previous work has demonstrated a significant inverse correlation between serum folate level and severity of negative symptoms in schizophrenia (Goff et al., Am J Psychiatry 161:1705-1708, 2004). A MTHFR 677C>T genotype, which confers an 222Ala>Val amino acid change, contributes to this relationship: patients who carry at least one copy of the 677T allele, which causes a 35 percent reduction in MTHFR activity (Frosst et al., Nat Genet 10:111-113, 1995), demonstrate greater negative symptom severity; among patients homozygous for the hypofunctional 677T allele, those who also have low serum folate are at especially high risk for negative symptoms (Roffman et al., Biol Psychiatry 63:42-48, 2008).

Dietary folate supplies the primary substrate for enzymes in the folate metabolic pathway, which in turn provides one-carbon moieties for DNA methylation, homocysteine metabolism, and other vital transmethylation reactions. Functional polymorphisms in the folate pathway influence the efficiency of downstream methylation events, and in the presence of reduced substrate, low-functioning genetic variants can become rate-limiting (Sharp and Little, Am J Epidemiol 159:423-443, 2004). For example, previous work with the MTHFR 677C>T variant indicated that among individuals homozygous for the fully functional C allele, genomic DNA methylation and homocysteine metabolism were not dependent on serum folate level; however, for individuals homozygous for the hypofunctional 677T variant, DNA methylation and homocysteine metabolism were strongly dependent on serum folate concentration (Friso et al., Proc Natl Acad Sci USA 99:5606-5611, 2002). Previously, an analogous pattern with respect to negative symptom severity in schizophrenia was reported, where folate levels influenced negative symptoms in T/T but not C/C patients (Roffman et al., Biol Psychiatry 63:42-48, 2008). Among T/T individuals, higher serum folate levels conferred DNA methylation patterns (Friso et al., Proc Natl Acad Sci USA 99:5606-5611, 2002) and negative symptom scores (Roffman et al., Biol Psychiatry 63:42-48, 2008) that did not differ substantially from C/C subjects, suggesting that T allele-related MTHFR dysfunction is surmountable in the presence of increased dietary folate.

FOLH1, also called GCP-II, is a glutamate carboxypeptidase that is anchored to the intestinal brush border, where it converts dietary polyglutamylated folates into monoglutamyl folates that can be transported into the body. The 484T>C variant is located in exon 2 of the structural transmembrane region and confers a 75Tyr>H is amino acid change. In a study of the Hordaland homocysteine cohort, Halsted and colleagues (Halsted et al., Am J Clin Nutr 86:514-521, 2007) reported elevated homocysteine among individuals with the C/T genotype, although there was no significant genotype effect on serum folate. Here, the 484C variant was associated with more severe negative symptoms. Of note, the 484T variant was associated with a decrease in negative symptoms in response to folate and/or vitamin B12 treatment. Folate hydrolase 1 is also expressed in the brain where it is known as NAALADase and cleaves n-acetylaspartylglutamate (NAAG) into n-acetylaspartate (NAA) and glutamate (Bacich et al., Mamm Genome 12:117-123, 2001). NAA is a marker of neuronal integrity for which hippocampal and prefrontal levels are consistently reduced in magnetic resonance spectroscopy studies of schizophrenia (Marenco et al., Adv Exp Med Biol 576:227-40, 2006), while glutamatergic dysfunction in schizophrenia is well established (Coyle J T, Cell Mol Neurobiol 26:365-384, 2006). FOLH1 therefore represents an important target in schizophrenia pathophysiology through its effects on numerous implicated pathways.

The 2756A variant of MTR has also been associated with elevated homocysteine levels compared to 2756G carriers in numerous studies (reviewed in Sharp and Little, Am J Epidemiol 159:423-443, 2004). Given that MTR remethylates homocysteine to methionine, homocysteine elevations in 2756A carriers suggest that this version of MTR confers reduced activity. Kempisty and colleagues (Kempisty et al., Psychiatr Genet 17:177-181, 2007) found that the MTR 2756G allele predicted increased risk of schizophrenia and bipolar disorder. In this study, however, it was the 2756A allele that appeared detrimental with respect to negative symptoms. MTR 2756A>G, which represents an amino acid change of 919Asp>Gly, is thus similar to MTHFR 677C>T, in that the allelic variant associated with reduced availability of one-carbon moieties is the same one that predicts greater negative symptom severity.

The present results extend previous MTHFR analyses to a larger cohort, confirming detrimental effects of the 677T variant on negative symptoms. As previously reported, no significant effect for the MTHFR 1298A>C polymorphism was found, which is also hypofunctional but not to the same degree as 677C>T (Lievers et al., J Mol Med 79:522-528, 2001). Both the 677T and 1298C alleles have been associated with significant increases in schizophrenia risk in a recent large meta-analysis using the SZGene database (Allen et al., the SzGene database. Nat Genet 40:827-834, 2008); however, as of September 2010, only the 677T allele remained significant in SZGene (N=4,362 patients and 5,840 controls; odds ratio 1.16; 95% confidence interval 1.05-1.27).

The COMT 675G>A variant, which has been consistently implicated in prefrontal function in brain imaging studies (Roffman et al., Harv Rev Psychiatry 14:78-91, 2006) but not in schizophrenia risk (Allen et al., the SzGene database. Nat Genet 40:827-834, 2008), was included in the regression analysis due to its previously reported interactive effects with MTHFR 677C>T on executive dysfunction (Roffman et al., Am J Med Genet B Neuropsychiatr Genet 147B:990-995, 2008) and related prefrontal impairment (Roffman et al., Proc Natl Acad Sci USA 105:17573-17578, 2008) in schizophrenia and on homocysteine metabolism (Tunbridge et al., Am J Med Genet B Neuropsychiatr Genet 147B:996-999, 2008). A trend-level, detrimental effect of the high activity COMT 675G allele on negative symptom severity was observed; however, COMT was not included in the follow-up risk allele analysis because it did not significantly predict negative symptoms in the present study.

Although common genetic variants may contribute approximately one-third of the total genetic liability in schizophrenia (Purcell et al., Nature 460:748-752, 2009), effects of individual variants are small, and many variants that show consistent replication in candidate gene studies are still not strong enough to reach genome-wide significance. Understanding how variants of small effect combine to exert clinically meaningful influences on schizophrenia phenotypes will be critical in deciphering the genetic architecture of the disorder. Increasingly, genome wide association studies and other high-throughput genetic investigations are relying on metabolic pathway analyses in order to pool risk variants into biologically meaningful contexts (Mill et al., Am J Hum Genet 82:696-711, 2008; O'Dushlaine et al., in press). Described herein are genetic variants across a single metabolic pathway and their contribution to negative symptom risk in schizophrenia. Subjects who possess a greater number of functional genetic variants in the folate pathway are particularly susceptible for negative symptoms, perhaps reflecting a cumulative effect of these variants on downstream methylation reactions. The approach of canvassing genetic variants in implicated biological pathways to generate cumulative risk scores holds promise in resolving the so-called “missing heritability” in schizophrenia and other complex genetic disorders in psychiatry (Maher, Nature 456:18-21, 2008) just as in the present study, where the net effects of folate-related variants outweigh the influence of a single variant on negative symptom severity.

Even among patients who carry multiple risk alleles, negative symptoms can be ameliorated in the presence of elevated serum folate levels.

### Schizophrenia

Schizophrenia is a chronic, severe, and disabling brain disease. Approximately 1-1.5 percent of the population develops schizophrenia during their lifetime; more than 2 million Americans suffer from the illness in a given year. Although schizophrenia affects men and women with equal frequency, the disorder often appears earlier in men, usually in the late teens or early twenties, than in women, who are generally affected in the twenties to early thirties. People with schizophrenia often suffer terrifying symptoms such as hearing internal voices not heard by others, or believing that other people are reading their minds, controlling their thoughts, or plotting to harm them. These symptoms may leave them fearful and withdrawn. Their speech and behavior can be so disorganized that they may be incomprehensible or frightening to others. Available treatments can relieve many symptoms, but most people with schizophrenia continue to suffer some symptoms throughout their lives; it has been estimated that no more than one in five individuals recovers completely.

The first signs of schizophrenia often appear as confusing, or even shocking, changes in behavior. Coping with the symptoms of schizophrenia can be especially difficult for family members who remember how involved or vivacious a person was before they became ill. The sudden onset of severe psychotic symptoms is referred to as an acute phase of schizophrenia. Psychosis, a common condition in schizophrenia, is a state of mental impairment marked by hallucinations, which are disturbances of sensory perception, and/or delusions, which are false yet strongly held personal beliefs that result from an inability to separate real from unreal experiences. Less obvious symptoms, such as social isolation or withdrawal, or unusual speech, thinking, or behavior, may precede, be seen along with, or follow the psychotic symptoms.

Some people have only one such psychotic episode; others have many episodes during a lifetime, but lead relatively normal lives during the interim periods. However, an individual with chronic schizophrenia, or a continuous or recurring pattern of illness, often does not fully recover normal functioning and typically requires long-term treatment, generally including medication, to control the symptoms.

Schizophrenia is found all over the world. The severity of the symptoms and long-lasting, chronic pattern of schizophrenia often cause a high degree of disability. Medications and other treatments for schizophrenia, when used regularly and as prescribed, can help reduce and control the distressing symptoms of the illness. However, some people are not greatly helped by available treatments or may prematurely discontinue treatment because of unpleasant side effects or other reasons. Even when treatment is effective, persisting consequences of the illness, lost opportunities, stigma, residual symptoms, and medication side effects may be very troubling.

### Example 1

Study procedures were approved by the Partners HealthCare and Massachusetts Department of Mental Health institutional review boards, and all participants provided written informed consent. Included in this study were 266 medicated, chronic schizophrenia outpatients (mean age 41±12 years, 70% male, 77% Caucasian) from an urban community mental health center clinic. A diagnosis of schizophrenia was confirmed by a consensus diagnostic conference based on results from a clinical diagnostic interview, chart review, and review of clinical history with treating physicians.

Patients were administered the Positive and Negative Syndrome Scale (PANSS) (Kay et al., Schizophrenia Bulletin 13:261-276) to assess symptom severity by trained raters who were blind to genotype and serum folate level.

DNA was obtained from blood samples and genotyped for six variants across five genes that regulate folate metabolism: FOLH1, RFC, MTHFR, MTR, and MTRR (Table 2). Specific variants were selected on the basis of (1) common occurrence in the general population (minor allele frequency>0.2), (2) coding for non-synonymous mutations in amino acid sequences, and (3) previous support in the literature for association with schizophrenia and/or measurable effects on folate or homocysteine metabolism. Patients were also genotyped for the COMT 675G>A polymorphism. No additional genetic variants were studied. Genotyping was conducted using the MASSARRAY® platform (Sequenom, San Diego, Calif.) using the nucleotide primers shown in Table 3.

Serum folate levels, obtained on the day of PANSS ratings, were available for a subset of 70 patients. Folate concentrations were determined using cloned enzyme donor immunoassay kits (BioRad, Hercules, Calif.) according to the manufacturer's instructions.

TABLE 2
Polymorphisms Across Genes that Regulate Folate Metabolism

| | | MAF | HWE | | HWE |
|---|---|---|---|---|---|
| | | (all | (all | MAF | (Cauca- |
| Gene | SNP | subjects) | subjects) | (Caucasians) | sians) |
| FOLH1 | rs202676 | 0.26 (C) | 0.90 | 0.19 (C) | 0.27 |
| | (484T > C) | | | | |
| RFC | rs1051266 | 0.48 (A) | 0.15 | 0.45 (A) | 0.03 |
| | (80A > G) | | | | |
| MTHFR | rs1801131 | 0.34 (C) | 0.26 | 0.31 (C) | 0.50 |
| | (1298A > C) | | | | |
| | rs1801133 | 0.31 (T) | 0.62 | 0.36 (T) | 0.81 |
| | (677C > T) | | | | |
| MTR | rs1805087 | 0.25 (G) | 0.01 | 0.20 (G) | 0.44 |
| | (2756A > G) | | | | |
| MTRR | rs1801394 | 0.43 (C) | 0.59 | 0.48 (C) | 0.55 |
| | (203A > G) | | | | |
| COMT | rs4680 | 0.46 (A) | 0.27 | 0.50 (G) | 0.67 |
| | (675G > A) | | | | |

MAF: minor allele frequency;
HWE: Hardy Weinberg equilibrium.

TABLE 3
Nucleotide Primers to Detect SNPs

| SNP | Forward Primer | Reverse Primer | Extension Primer |
|---|---|---|---|
| rs1801133 | ACGTTGGATGGAAG | ACGTTGGATGAGCCT | AAGGTGTCTGCGGGAG |
| | CACTTGAAGGAGAA | CAAAGAAAAGCTGCG | (SEQ ID NO: 6) |
| | GG (SEQ ID NO: 4) | (SEQ ID NO: 5) | |
| rs1805087 | ACGTTGGATGCTTT | ACGTTGGATGTCTAC | AGAATATGAAGATAT |
| | GAGGAAATCATGGA | CACTTACCTTGAGAG | TAGACAGG |
| | AG (SEQ ID NO: 7) | (SEQ ID NO: 8) | (SEQ ID NO: 9) |
| rs202676 | ACGTTGGATGCTTT | ACGTTGGATGGTCCA | TAAAGCTGAGAACATC |
| | GAGGAAATCATGGA | TATAAACTTTCGAGG | AAGAAGTTCTTA |
| | AG (SEQ ID NO: 10) | (SEQ ID NO: 11) | (SEQ ID NO: 12) |

Results

SNPs Predictive of Negative Symptoms

A multiple linear regression model was used to determine independent effects of each of the seven SNPs on the PANSS negative symptom subscale. For each SNP, genotype was entered as 0, 1, or 2, depending on the number of minor alleles; the regression model determined whether risk allele load significantly (two-tailed p<0.05) predicted negative symptom severity. All SNP variables were entered simultaneously into the model. An identical analysis was attempted for the PANSS positive symptom subscale.

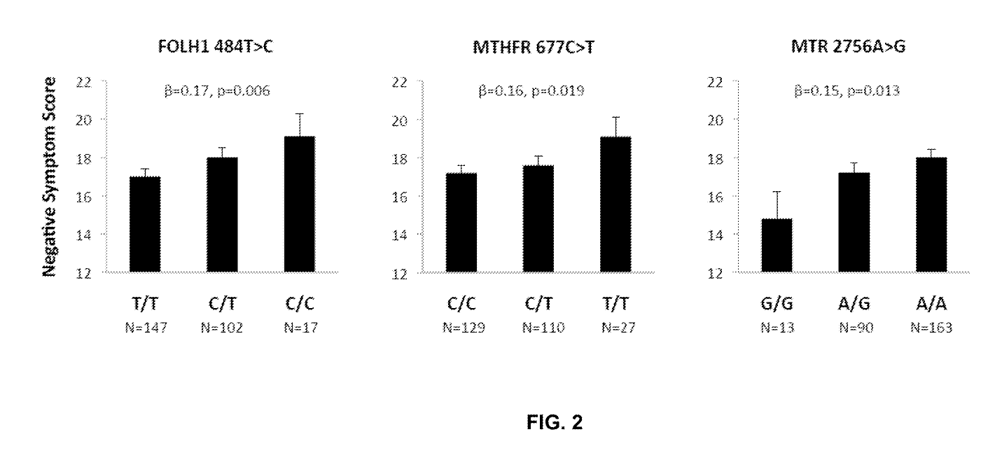

Regression analyses are reported in Table 4 and FIG. 2. For negative symptoms, as previously reported in a smaller version of the same cohort (n=200) (Roffman et al., 2008), MTHFR 677T allele load was significantly associated with negative symptom severity. In addition, FOLH1 484C allele load and MTR 2756A allele load predicted negative symptom scores. COMT 675G allele load was associated with negative symptom scores at trend level. The model accounted for 7.2% of the variance in negative symptoms (R²). In contrast, none of the polymorphisms studied were significantly associated with positive symptom scores.

Regression analyses were repeated using only subjects with self-reported Caucasian race (n=204) to examine the possibility of stratification artifact. Negative symptom results remained statistically significant using the Caucasian subsample (Table 4), wherein the model accounted for 10.4% of the variance.

TABLE 4
Independent Effects of Seven Single Nucleotide Polymorphisms on
PANSS Negative and Positive Symptoms

| | | | | | Caucasian subjects | | |
|---|---|---|---|---|---|---|---|
| | | All subjects (n = 266) | | | (n = 204) | | |
| Gene | SNP | Beta | t | p | Beta | t | p |

PANSS Negative Symptoms

| | | | | | | | |
|---|---|---|---|---|---|---|---|
| FOLH1 | rs202676 | 0.172 | 2.74 | 0.006 | 0.135 | 1.97 | 0.050 |
| | (484T > C) | (C) | | | (C) | | |
| RFC | rs1051266 | 0.028 | 0.47 | 0.641 | 0.017 | 0.26 | 0.798 |
| | (80A > G) | | | | | | |
| MTHFR | rs1801131 | 0.074 | 1.10 | 0.273 | 0.109 | 1.36 | 0.177 |
| | (1298A > C) | | | | | | |
| | rs1801133 | 0.164 | 2.37 | 0.019 | 0.202 | 2.53 | 0.012 |
| | (677C > T) | (T) | | | (T) | | |
| MTR | rs1805087 | 0.151 | 2.50 | 0.013 | 0.216 | 3.16 | 0.002 |
| | (2756A > G) | (A) | | | (A) | | |
| MTRR | rs1801394 | 0.076 | 1.23 | 0.220 | 0.021 | 0.30 | 0.763 |
| | (203A > G) | | | | | | |
| COMT | rs4680 | 0.112 | 1.81 | 0.071 | 0.150 | 2.17 | 0.031 |
| | (675G > A) | (G) | | | (G) | | |

Overall model statistics: For all subjects, R² = 0.072, adjusted R² = 0.047,
F(7,265) = 2.86, p = 0.007; For Caucasian subjects, R² = 0.104,
adjusted R² = 0.072, F(7,203) = 3.24, p = 0.003.

PANSS Positive Symptoms

| | | | | | | | |
|---|---|---|---|---|---|---|---|
| FOLH1 | rs202676 | 0.048 | 0.76 | 0.448 | 0.137 | 1.96 | 0.052 |
| | | | | | (T) | | |
| RFC | rs1051266 | 0.045 | 0.73 | 0.466 | 0.005 | 0.07 | 0.944 |
| MTHFR | rs1801131 | 0.087 | 1.26 | 0.209 | 0.146 | 1.77 | 0.078 |
| | | | | | (C) | | |
| | rs1801133 | 0.095 | 1.34 | 0.181 | 0.076 | 0.92 | 0.357 |
| MTR | rs1805087 | 0.055 | 0.90 | 0.369 | 0.071 | 1.01 | 0.313 |
| MTRR | rs1801394 | 0.049 | 0.78 | 0.436 | 0.034 | 0.48 | 0.629 |
| COMT | rs4680 | 0.068 | 1.08 | 0.280 | 0.089 | 1.26 | 0.209 |

Overall model statistics:
For all subjects, R² = 0.033, adjusted R² = 0.006, F(7,265) = 1.24, p = 0.28; For Caucasian subjects, R² = 0.060, adjusted R² = 0.027, F(7,203) = 1.79, p = 0.091.

For significant or trend-level SNPs, the risk allele is given in parentheses next to the beta statistic.

### Cumulative Effects of Risk SNPs

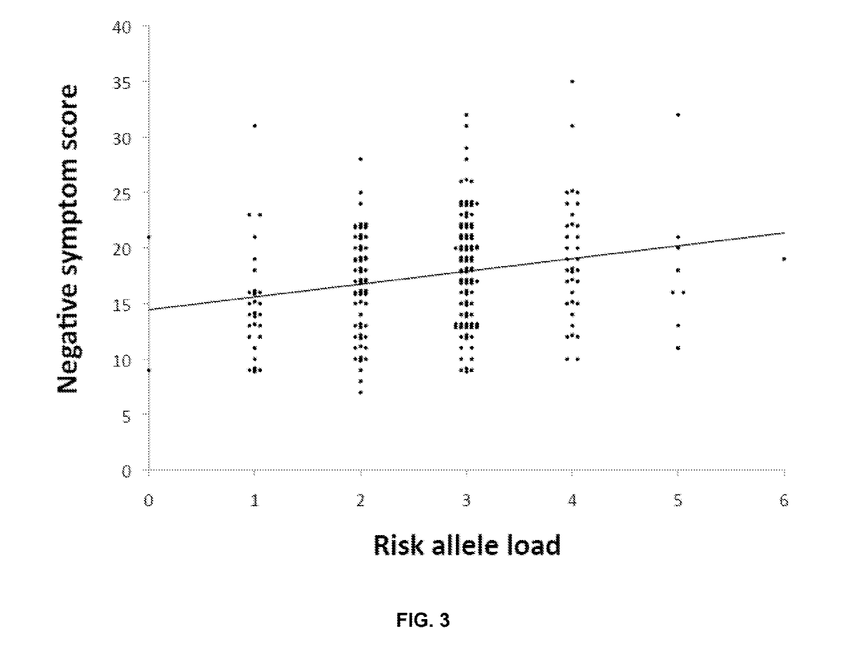

To illustrate more directly the cumulative effects of the identified risk SNPs on negative symptoms, subjects were assigned to groups based on the total number of risk alleles (i.e., (0, 1, or 2 copies of MTHFR 677T)+(0, 1, or 2 copies of FOLH1 484C)+(0, 1, or 2 copies of MTR 2756A)=0 to 6 total risk alleles). The relationship between negative symptoms and total risk allelic load is plotted in FIG. 3. The risk allele load model predicted negative symptoms equally well as a linear regression model where the three SNPs were entered separately (F(2,262)=0.10, p=0.90; R²=0.052 for three SNP regression model and R²=0.051 for the additive model). The three-SNP linear regression model fit the negative symptom data significantly better than MTHFR 677C>T alone (F(2,262)=5.7, p=0.004; for MTHFR alone, R²=0.01).

Interaction with Folate

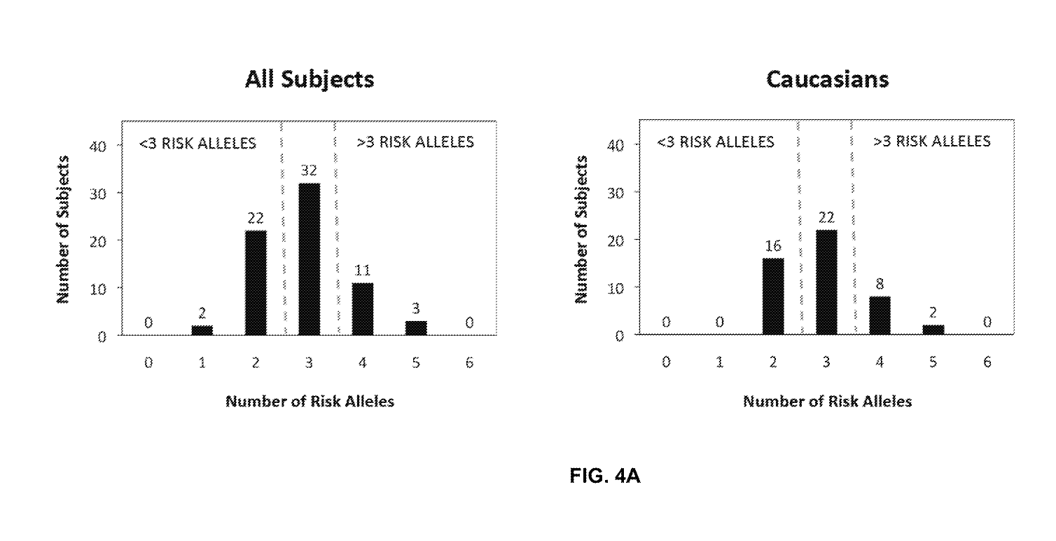

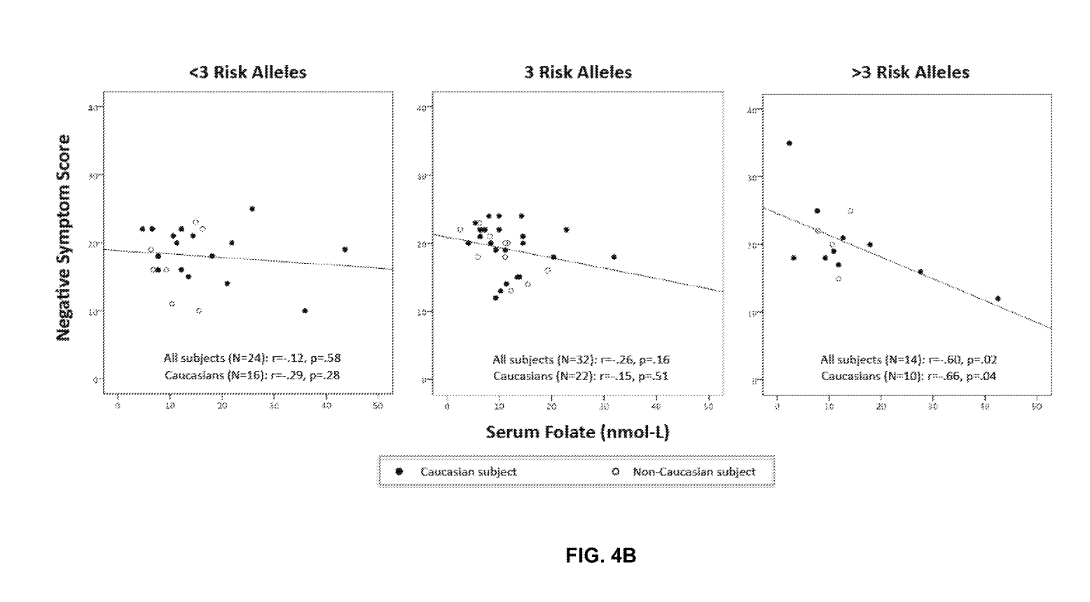

An exploratory analysis examining the relationship between risk allele load, negative symptoms, and folate level was conducted among participants for whom serum folate level was available. Subjects were divided into three groups based on the distribution of subjects by risk allele status (FIG. 4A): less than three risk alleles (n=24), three risk alleles (n=32), or greater than three risk alleles (n=14). Linear regression indicated an interaction between risk allele load and serum folate on negative symptom score (overall model p=0.005, R²=0.18; interaction, (β=−0.61, p=0.05).

Post hoc correlations between negative symptom scores and serum folate level were attempted separately for each group (FIG. 4B). For subjects with less than three or three risk alleles, the relationship between negative symptoms and serum folate was not significant. Conversely, for subjects with greater than three risk alleles, negative symptom severity was inversely correlated with serum folate level.

None of the genes by themselves or in any combination influence serum folate levels; the only one that might be expected to is FOLH1, since it translocates dietary folate through the gut into the bloodstream. MTR and MTHFR are intracellular, and thus downstream of serum folate.

Only the three-SNP model had a significant bearing on the folate-negative symptom relationship, while none of the two-SNP models reached statistical significance. The strength of the correlation between negative symptoms and “risk score” across all three polymorphisms (i.e., a score of 0 to 6 for each subject) and across two polymorphisms at a time (i.e., a score of 0 to 4 for each subject) was compared. The correlation was stronger for the three polymorphism model (R²=0.226, p=0.0002) than for any of the two polymorphism models (R²=0.164 to 0.186, p=0.002 to 0.007).

Correlations of risk allele load and negative symptoms for the various combinations of two- and three-SNPs are presented below:

MTHFR+MTR:R²=0.180,p=0.003

MTHFR+FOLH1:R²=0.164,p=0.007

MTR+FOLH1:R²=0.186,p=0.002

MTHFR+MTR+FOLH1:R²=0.226,p=0.0002

No significant correlation between serum folate and negative symptoms was observed for any of the two polymorphism models, for patients who had (a) 0-1, (b)₂, or (c) 3-4 copies of the risk alleles. However, with the three polymorphism model, there was a significant relationship in the group of patients who had 4-6 copies of risk alleles.

### Example 2

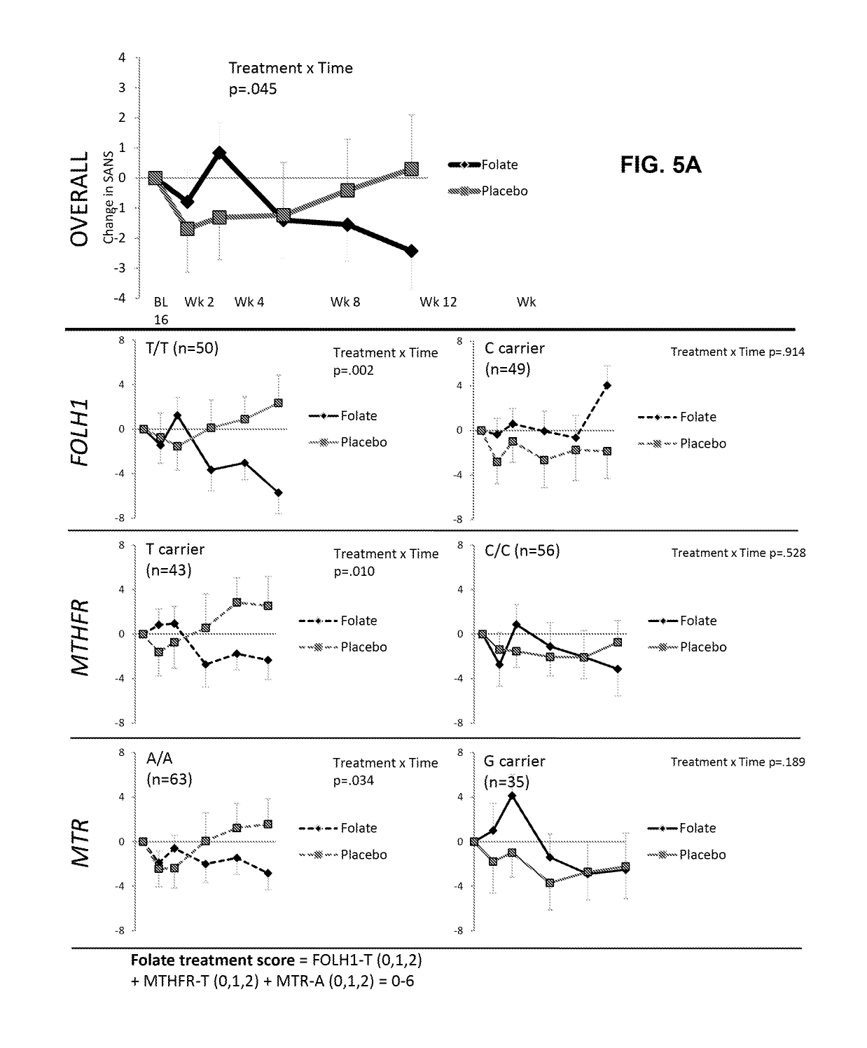

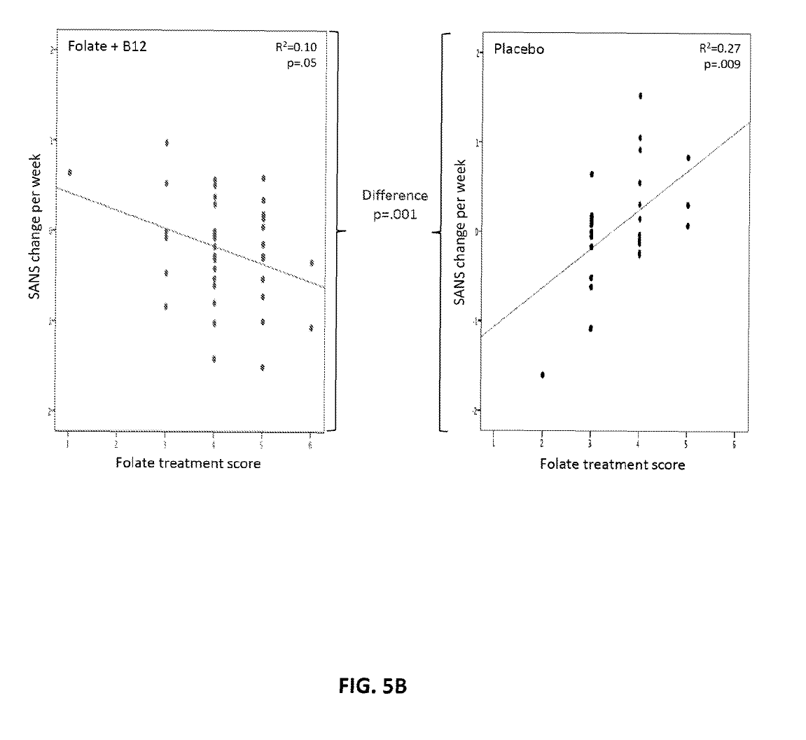

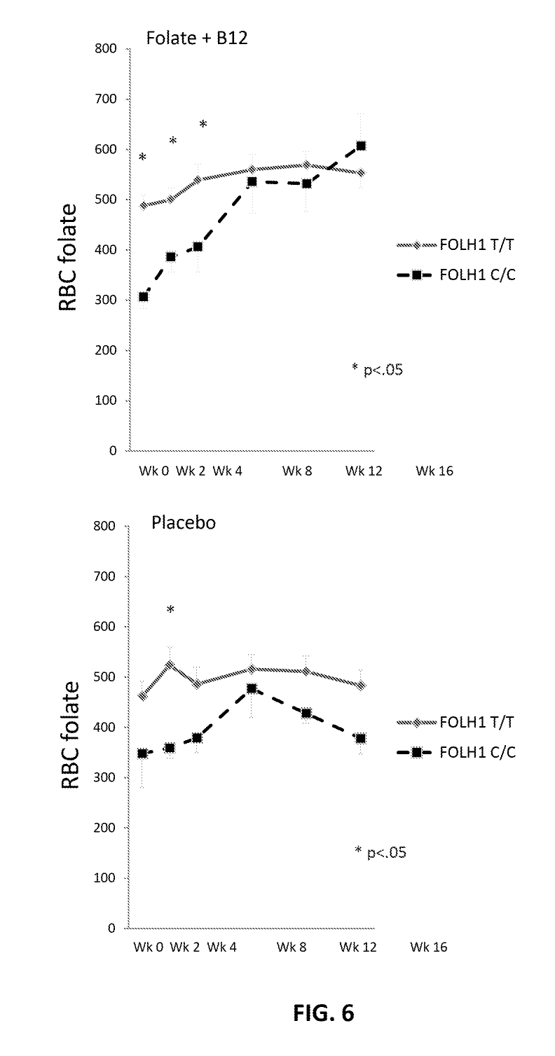

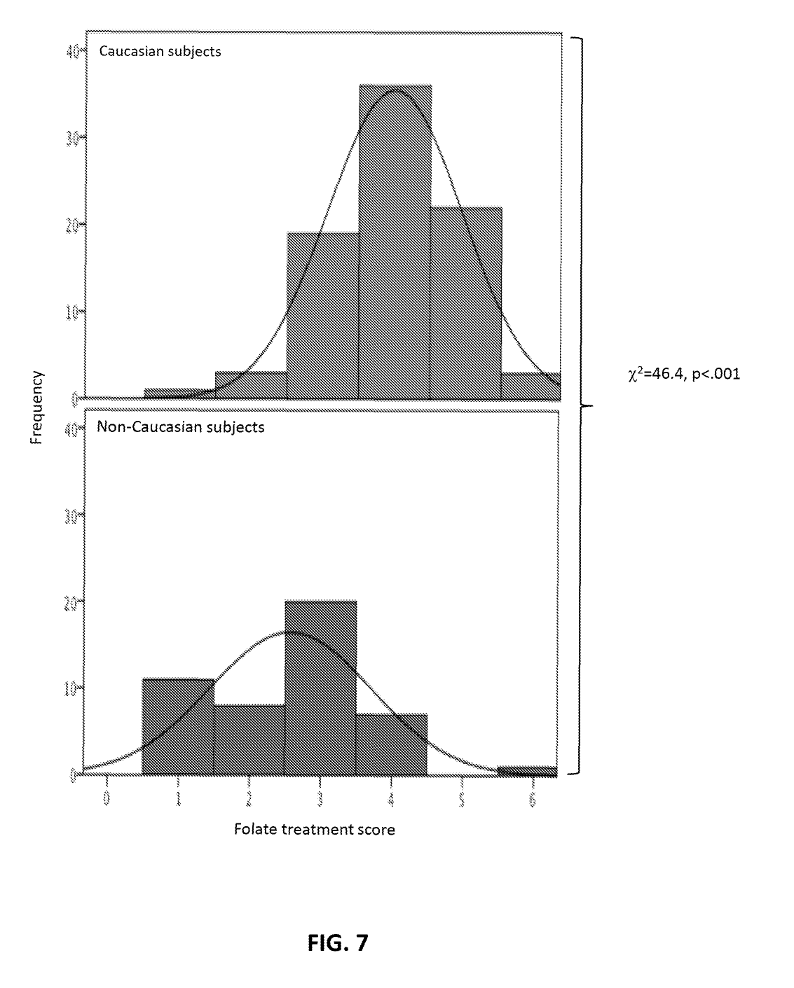

A three-site, placebo-controlled, double-blind trial of 16 weeks of daily 2 mg folic acid+400 mcg vitamin B12 supplements for negative symptoms of schizophrenia was performed with 140 randomized patients with chronic schizophrenia; 78% completed the trial. A significant overall benefit was observed for the group treated with folate and vitamin B12 compared to placebo. For each of the three SNPs, there was a difference in folate+vitamin B12 response depending on genotype. For the MTHFR (rs1801133) and MTR (rs1805087) SNPs, patients who carried the folate alleles (MTHFR 677T and MTR 2756A) showed significantly better response to folate than to placebo, while there was no treatment effect for patients who carried the normal versions of these genes (FIG. 5A). For FOLH1 (rs202676), the opposite was true: patients who carried the low-functioning variant (FOLH1 484C), which was also associated with a delayed increase in red blood cell folate elevation over the course of the trial (FIG. 6), did not show a beneficial effect of folate over placebo, while patients who carried the normal version showed a beneficial effect (FIG. 5A).

However, when these three SNPs were pooled together to create a folate treatment score (i.e., (0, 1, or 2 copies of MTHFR 677T)+(0, 1, or 2 copies of FOLH1 484T)+(0, 1, or 2 copies of MTR 2756A)=0 to 6 total folate alleles), there was a significant correlation between folate alleles and treatment response, where a higher folate treatment score was associated with better response to folate+vitamin B12 and a greater reduction in negative symptoms (FIG. 5B). This relationship was not found for the placebo group, and there was a significant treatment×risk score interaction (FIG. 5B). Among patients receiving placebo, a higher folate treatment score was associated with increased negative symptoms over the treatment period (FIG. 5B). Subjects who received a placebo capsule also participated in frequent interactions with study staff, including interviews and testing over a period of weeks or months, subjects with a low risk score calculated by the six folate alleles displayed an improvement in negative symptoms whereas subjects with high risk scores improved only with folate treatment. This demonstrates that an individual's likelihood of response to treatment with folate+vitamin B12 can be predicted by the six folate alleles.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method of treating a subject diagnosed as having a negative symptom of schizophrenia, the method comprising:
determining the presence of one or more alleles at rs1801133, rs1805087, and rs202676 in a sample comprising genomic DNA from the subject;
selecting a treatment for the subject based on the presence of the one or more alleles; and
treating the subject with the selected treatment.

2. The method of claim 1, wherein the method comprises determining the presence of six alleles, wherein the six alleles consist of two alleles at each of rs1801133, rs1805087, and rs202676.

3. The method of claim 2, wherein if a “T” at rs1801133, an “A” at rs1805087, and a “T” at rs202676 are present, and one or more additional alleles are a “T” at rs1801133, an “A” at rs1805087, or a “T” at rs202676, then a treatment comprising prescribing or administering folate to the subject is selected.

4. The method of claim 2, wherein if two alleles are a “T” at rs1801133 and two alleles are an “A” at rs1805087, then a treatment comprising prescribing or administering folate to the subject is selected.

5. The method of claim 4, wherein if one or more additional alleles is a “T” at rs202676, then a treatment comprising prescribing or administering folate to the subject is selected.

6. The method of claim 2, wherein if two alleles are a “T” at rs1801133, two alleles are an “A” at rs1805087, and two alleles are a “C” at rs202676, then a treatment comprising prescribing or administering folate to the subject is selected.

7. The method of claim 1, wherein the negative symptom is selected from the group consisting of apathy, impoverished speech, flattened affect, and social withdrawal.

8. The method of claim 1, wherein the selected treatment comprises prescribing or administering folate to the subject.

9. The method of claim 8, wherein the selected treatment further comprises prescribing or administering vitamin B12 to the subject.

10. The method of claim 2, wherein if a “T” at rs1801133, an “A” at rs1805087, or a “T” at rs202676 is not present, then a treatment comprising a psychosocial intervention is selected.

11. A method comprising:
assaying for the presence of one or more alleles at rs1801133, rs1805087, and rs202676 in a biological sample comprising genomic DNA from a subject diagnosed as having a negative symptom of schizophrenia; and
transmitting to a recipient a report on the presence of the one or more alleles.

12. The method of claim 11, wherein the method further comprises selecting a treatment for reducing the negative symptom in the subject based on the presence of the one or more alleles.

13. The method of claim 11, wherein the method comprises assaying for the presence of six alleles, wherein the six alleles consist of two alleles at each of rs1801133, rs1805087, and rs202676.

14. The method of claim 13, wherein if a “T” at rs1801133, an “A” at rs1805087, and a “T” at rs202676 are present, and one or more additional alleles are a “T” at rs1801133, an “A” at rs1805087, or a “T” at rs202676, then a treatment comprising prescribing or administering folate to the subject is selected.

15. The method of claim 13, wherein if two alleles are a “T” at rs1801133 and two alleles are an “A” at rs1805087, then a treatment comprising prescribing or administering folate to the subject is selected.

16. The method of claim 15, wherein if one or more additional alleles is a “T” at rs202676, then a treatment comprising prescribing or administering folate to the subject is selected.

17. The method of claim 13, wherein if two alleles are a “T” at rs1801133, two alleles are an “A” at rs1805087, and two alleles are a “C” at rs202676, then a treatment comprising prescribing or administering folate to the subject is selected.

18. The method of claim 11, wherein the negative symptom is selected from the group consisting of apathy, impoverished speech, flattened affect, and social withdrawal.

19. The method of claim 12, wherein the selected treatment comprises prescribing or administering folate to the subject.

20. The method of claim 19, wherein the selected treatment further comprises prescribing or administering vitamin B12 to the subject.
